(19) United States
(12) Patent Application Publication
(10) Pub. No.: US 2008/0004637 A1
(43) Pub. Date: Jan. 3, 2008

(54) SURGICAL CLIP, APPLICATOR AND APPLICATOR METHODS

(76) Inventors: James B. Klassen, Langley (CA); David W. Boehm, Summerland (CA)

Correspondence Address: DYKAS, SHAVER & NIPPER, LLP, P.O. BOX 877, BOISE, ID, 83701-0877, US

(21) Appl. No.: 11/742,259

(22) Filed: Apr. 30, 2007

Related U.S. Application Data
(60) Provisional application No. 60/746,137, filed on May 1, 2006.
(60) Provisional application No. 60/746,034, filed on Apr. 29, 2006.

(51) Int. Cl. A61B 17/10; A61B 17/08

(52) U.S. Cl. 606/142; 606/157

(57) ABSTRACT

A surgical clip according to an embodiment includes a pair of jaws, pivoted together, with a spring holding the jaws together. Various design embodiments incorporate this structure and include additional features such as having jaws made of an MRI invisible material, and spring connected to the jaws with hinge joints. An applicator is also provided, particularly with a ball and socket connection to the surgical clip. A method of latching the applicator, and of handling the clip with the applicator are also provided.

### BACKGROUND

Clips are used to occlude vessels and aneurysms in a person's brain or spinal cord. These clips are typically called aneurysm clips and may be made for example of titanium or cobalt alloy with jaws integrally formed with a coiled spring. Examples are the Sugita™ brand of aneurysm clips available from Mizuho America, Inc., Beverly, Mass. 10915 USA, and aneurysm clips made by Aesculap Inc. of Center Valley, Pa. 18034 USA. While these clips are quite well used, the use of metal alloy in the jaws of the clips means that the clips interfere with magnetic or X-ray imaging processes in particular determining with MR or CT imaging residual or recurrent aneurysm neck i.e. incomplete obliteration. This is important as aneurysms not completely obliterated are at risk for growth and rupture. In addition, the design has a tendency to scissoring, which runs the risk of cutting into a vessel or incompletely obliterating the aneurysm. While ceramic clips have been proposed as for example in EP0346084, they are not commonly used. Applicators, available from Mizuho and Aesculap, for manipulating and placing the clips are also known.

### SUMMARY

A variety of surgical clips and applicators are disclosed, particularly for use in occluding vessels and aneurysms.

A surgical clip according to an embodiment includes a pair of jaws, pivoted together, with a spring holding the jaws together. Various design embodiments incorporate this structure and include additional features. Some designs of the surgical clips allow use of MRI invisible material such as silicon nitride ceramic for the jaws. Use of MRI invisible materials makes these designs particularly suitable for use in MRI or CT applications. Some of the designs also are made with titanium alloy, which has excellent biocompatibility, strength, elasticity and MRI compatibility. Titanium alloy is not MRI invisible, so in some designs, titanium alloy components such as a spring are positioned away from the clamping faces of the jaws where the aneurysm is occluded. Positioning the spring away from the clamping faces and close to the pivot also provides the clip with tremendous stability by virtue of the high spring force that is required with the spring such a short distance from the pivot. This stability is also enhanced by the hinge joint of the spring and other factors described in this patent document which prevent the jaws from scissoring laterally or disassembling under normal use.

Additional clip features include for example one or more of the following features: the spring contacting the jaws at hinge joints; the hinge joints having confining walls with sloped contact faces at various angles; contact points of the spring with the jaws being spatially distributed; a tongue and groove structure in the hinge joints for centering; the spring ends to pivot angle being large; a center gap in the hinge joint between spring ends and the jaws, an offset rear portion (applicator end) of the spring for receiving an applicator, the spring side surface providing a locating surface for applicator tips; ball and socket connection to an applicator; a flange at the back of the applicator end; the jaws being formed of an MRI invisible material and the spring being made of an MRI compatible metal, the applicator ends may have matching surfaces that contact each other when the jaws are fully opened, as for example being flat surfaces; one or both hinge joints at an angle, for example more than zero but less than 30 degrees, to pivot axis; wide hinge joints and pivot for stability; parallel jaws when separated; divergent, arched, polygonal or circular jaws; T or L or stitch shaped jaws; curved jaws; jaws extending radially from a circular or polygonal jaw portion; jaws with clamping faces that are off a center plane of the clip; the spring is constructed to provide a uniform spring opening resisting force across the spring ends; the jaws are gray and the spring is a different color and material from the jaws; and at least a portion of at least one of the clamping faces of the jaws is provided with a variety of structured surfaces, for example an interrupted structured surface.

A medical applicator according to an embodiment has a pair of arms connected by a pivot having a pivot axis, each arm of the pair of arms having a tip end on one side of the pivot and a handle on the other side of the pivot, and a spring between the arms, the spring being attached to at least one of the arms. Various design embodiments incorporate this structure and include additional features for example one or more of the following features: when used in combination with a clip having jaws, a side face of jaw, at least near the pivot of the clip, is coplanar (flush) with a side face of the applicator, at least up to the pivot of the applicator; each handle having an inner portion facing the other handle, the inner portions incorporating a grip surface; a latch secured to one handle by a latch pivot, a catch on the other handle, the latch pivot extending fully across the one handle to resist bending of the latch; a latch secured to a first one of the handles by a latch pivot, a catch on the second one of the handles, the latch being biased by a latch spring towards the first handle; the latch has a latching end with a hook, the hook having a slanted inwardly widening exterior face; one or more stops preventing contact of the latching end of the latch with the first handle and/or preventing rotation of the latch by more than a pre-defined distance beyond the catch and the tip ends are formed as either a ball or socket.

A method of latching a medical applicator is also provided where the medical applicator has handles separated by a spring and latch, and the steps of the method include closing the handles against force provided by the applicator spring (or the applicator spring and the clip spring), moving the latch against force provided by a latch spring until a hook on the latch abuts against a catch and continuing to close the handles together while pressing the hook against the catch until the hook latches onto the catch.

These and other aspects of the device and method are set out in the claims, which are incorporated here by reference.

### BRIEF DESCRIPTION OF THE FIGURES

Embodiments will now be described with reference to the figures, in which like reference characters denote like elements, by way of example, and in which:

FIG. 1 is a side view of an embodiment of a clip;

FIG. 2 is a perspective view of an embodiment of the clip of FIG. 1;

FIG. 3 is a plan view of the clip of FIG. 1;

FIG. 4 is a side view of the clip of FIG. 1 in open position with applicator ends contacting each other over a wide area;

FIG. 5 is a perspective view of the clip of FIG. 1 in the position of FIG. 4;

FIG. 6 is an enlarged side view of the clip of FIG. 1;

FIG. 7 is an enlarged side view of a part of the jaw of the clip of FIG. 1;

FIG. 8 is a perspective view of the part of the clip shown in FIG. 7;

FIG. 9 is an enlarged side view of a hinge joint of the clip of FIG. 1;

FIG. 10 is an enlarged side view of a pivot-jaw contact of the clip of FIG. 1;

FIG. 11 is a side view showing spacing of parallel clamping faces of the jaws of the clip of FIG. 1;

FIG. 12 is a side view showing diverging jaw faces of the clip of FIG. 1;

FIG. 13 is a perspective view of an embodiment of an applicator;

FIG. 14 is a side of the applicator of FIG. 13;

FIG. 15 is a perspective view of a tip end of the applicator of FIG. 13;

FIG. 16 is a top view of the tip end of the applicator of FIG. 13;

FIG. 17 is a bottom view of the tip end of the applicator of FIG. 13;

FIG. 18 is an enlarged view of the applicator of FIG. 13 showing inside grip portions of the handles;

FIG. 19 is a top view of a latch portion of the applicator of FIG. 13;

FIG. 20 is a further view of the applicator of FIG. 13;

FIG. 21 is an enlarged view of the latch of the applicator of FIG. 13 in stowed position;

FIG. 22 is an enlarged view of the latch pivot of the applicator of FIG. 13 with the latch in open position;

FIG. 23 is an enlarged view of the latch pivot of the applicator of FIG. 13 with the latch in about to latch position;

FIG. 24 is an enlarged view of the latch pivot of the applicator of FIG. 13 with the latch in latched position;

FIG. 25 is a perspective view offset from the rear of the clip of FIG. 1 held by the applicator of FIG. 13;

FIG. 26 is a top view of the clip of FIG. 1 held by the applicator of FIG. 13;

FIG. 27 is a perspective view of the clip of FIG. 1 held by the applicator of FIG. 13 at an angle;

FIG. 28 is a top view of the clip of FIG. 1 held by the applicator of FIG. 13 at an angle;

FIGS. 29-31 are perspective views of clips with straight jaws with circular portions;

FIGS. 32-34 are top views respectively of the clips of FIGS. 29-31;

FIG. 35 is a side view of the clips of FIGS. 29-31;

FIG. 36 is a side view of three clips with straight jaws and different lengths;

FIGS. 37-39 are top views of the clips of FIG. 36;

FIGS. 40-42 are perspective views of the clips of FIG. 36;

FIGS. 43-44 are perspective views of clips with curved jaws;

FIG. 45 is a side view of the clips of FIGS. 43-44;

FIGS. 46-47 are respectively top views of the clips of FIGS. 43-44;

FIG. 48 is a side view of a clip with a circular jaw portion and a clamping section forming arms of a T with respect to the remainder of the jaws;

FIG. 49 is a perspective view of the clip of FIG. 48;

FIG. 50 is a top view of the clip of FIG. 48;

FIG. 51 is a perspective view of a clip with a circular jaw portion and a clamping jaw section forming an arm of an L with respect to the remainder of the jaws;

FIG. 52 is a top view of the jaws of FIG. 51;

FIGS. 53-55 are respectively a side, top and perspective view of a clip with circular jaw portion and a clamping section offset from a plane bisecting the pivot and spring, the clamping section forming a T shape with respect to the remainder of the jaws;

FIGS. 56-57 are respectively a side view and perspective view of a clip with circular jaw portion and clamping section extending radially from the circular jaw portion;

FIGS. 58-59 are perspective views of clips on an aneurysm and vessel;

FIGS. 60-62 are respectively a side view, top view and perspective view of a jaw with circular jaw portion and an L shaped clamping section offset from the remainder of the jaws;

FIGS. 63-65 are respectively a perspective view, top view and side view of an applicator that may be used with a clip having a rear flange;

FIGS. 66-70 are respectively a side view pre-engaging position, side view engaged position, side view jaw opening position, first perspective view pre-engaging position and second perspective view pre-engaging position of a clip with rear flange and the applicator of FIGS. 63-65;

FIG. 71 is a perspective view of part of an embodiment of an applicator;

FIG. 72 is a perspective view of the clip of FIG. 1 with applicator ends configured to receive the applicator of FIG. 71;

FIG. 73 shows a clip and applicator combination with ball and socket clip to applicator contact surfaces;

FIG. 74 shows a clip and applicator combination with conicial clip to applicator contact surfaces;

FIG. 75 is a perspective view of a clip with integral pivot;

FIGS. 76-80 show various embodiments of jaws with integral pivots;

FIGS. 81-85 are various views of pivots;

FIGS. 86-89 show an embodiment of a spring for use with various clips;

FIGS. 90-91 show a further embodiment of a spring for use with various clips;

FIGS. 92-94 show a further embodiment of a spring for use with various clips;

FIGS. 95-97 show a further embodiment of a spring for use with various clips;

FIGS. 98-100 show embodiments of a spring with integral pivot;

FIGS. 101-102 show embodiments of a spring with constant closing force across the spring ends;

FIGS. 103-105 are side views of clips showing jaw opening angles;

FIGS. 106-107 are top view of clips showing variable positioning of a spring hinge;

FIGS. 108-110 show surface structure on jaw clamping faces;

FIGS. 111-113 are respectively a top, side and perspective view of a jaw with straight jaws terminating in a T clamping section;

FIGS. 114-116 are respectively a top, side and perspective view of a jaw with straight jaws terminating in an L shaped clamping section;

FIGS. 117-119 are respectively a side, top and perspective view of a clip with offset jaw cooperating with a straight jaw;

FIG. 120 is a further embodiment of an applicator with corresponding clip;

FIG. 121 shows a clip with divergent non-circular jaws; and

FIG. 122 shows a clip with hinge axis not parallel to the pivot axis.

### DETAILED DESCRIPTION

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims. The term “surgical” before clip means, at least in part, that the clip is made from bio-compatible materials suitable for short or long term use within a human.

The clips and applicators described here have applicability for medical purposes such as aneurysm occlusion, vascular occlusion (either arterial or venous) in the management of arteriovenous malformation (AVM) and temporary vascular occlusion such as during the management of extra- or intracranial occlusive vascular disease and central nervous system (CNS) neoplasia.

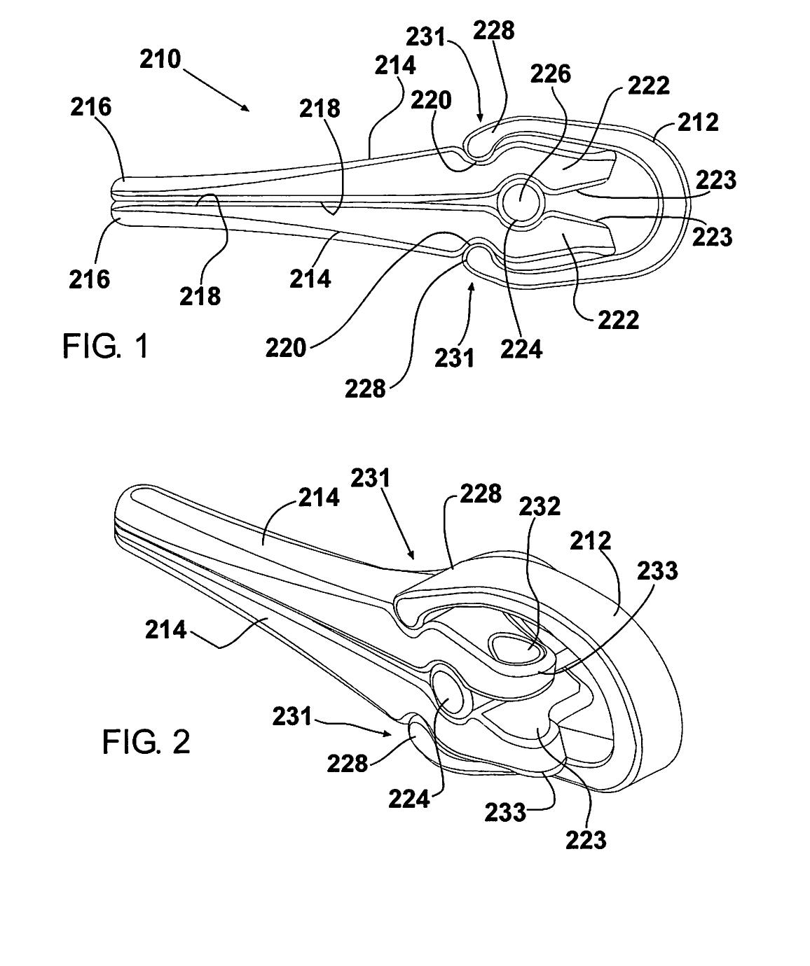

As shown in FIGS. 1-3, an embodiment of a surgical clip 210 is formed of a spring 212 that presses on a pair of jaws 214. Each jaw 214 has a clamping end 216, a clamping face 218, a socket 220 and an applicator end 222. The jaws 214 pivot about a pivot 224 having a pivot axis 226, which in FIG. 1 is perpendicular to the plane of the figure. The spring 212 has spring ends 228 which bear against a corresponding one of the jaws 214 between the clamping end 216 of the jaw and the pivot 224. The spring ends 228 are received in the sockets 220. In the embodiment shown, each contact of the spring ends 228 with the jaws 214 forms a hinge joint 231. The surgical clip 210 may for example have a length of about 20 mm and a jaw width of 1.5 mm, the size depending on the intended application. The closing force may be, for example 150 grams, measured 2.5 mm from the tip of the jaws. Significantly smaller clips and significantly larger clips are also possible.

As illustrated in FIG. 4, compression of the applicator ends 222 together causes the jaws 214 to open against the force of the spring 212. The rear faces 223 of the applicator ends 222 may be made to match each other so that they provide a large contact area. In the example of FIG. 4, the rear faces 223 are flat. Matching contact faces of the applicator ends 222 assist in preventing damage to the jaws 214 and reduce bending loads. As the jaws 214 open, the spring ends 228 rotate in the socket 220 about hinge axis 230. Due to the hinge nature of the joint between the jaws 214 and the spring 212, the rotation of the spring ends 228 in relation to the jaws 214 is confined. That is, the hinge joint 231 prevents twisting about an angle perpendicular to the hinge axis. The direction that the clip ends 228 move due to opening of the jaws 214 is perpendicular to the hinge axis 231. As shown, the movement of the spring ends 228 is perpendicular to the pivot axis, that is, the hinge axis 230 is parallel to the pivot axis 226. In another embodiment, the hinge axis may be at an angle to the pivot axis 226 as shown in clip 217 in FIG. 122 where hinge axis 229 of spring 213 on jaws 215 is at a non-zero angle to the pivot axis 226. Such a design helps to provide a more even force on the jaws when an offset spring is used. The angle shown could also be in the other direction (counterclockwise instead of clockwise rotation of the hinge axis as shown) and the angles of the hinge axes on both sides need not be equal. Angles up to 45 degrees are possible, though this is extreme, and angles up to 30 degrees are more reasonable. The hinge axis 230 strongly resists scissoring of the clip 210 under normal operation, and prevents disassembly under normal operation. The high spring force of the spring 212 which is necessary with a high hinge axis to pivot axis angle, also increases clip stability by securely clamping the jaws against the pivot. The offset portion of the spring 212 also provides a side surface facing the applicator ends 232 that allows a surgeon to more readily locate the applicator ends 232 of the clip 210.

As shown in FIGS. 2, 3 and 5, each applicator end 222 has an applicator contact area 232. In the embodiment shown, the applicator contact area 232 is formed as a socket having the shape of a spherical depression, but may have other configurations in some embodiments. For example, the applicator contact area 232 may be formed as a cone, or as a ball or ridge. An offset portion 234 of the spring 212 between the hinge joints 231 is offset from planes perpendicular to the pivot axis 226 that pass through the applicator contact areas 232. The offset portion 234 may comprise the entire back end of the spring 212. The offset portion 234 is offset sufficiently to allow the applicator ends 222 to be visible and accessible by an applicator (described below) from directly behind the surgical clip 210. The spring 212 is thus in this embodiment asymmetrical, which greatly eases the position of applicators on the clip. A ball and socket configuration for the applicator contact area 232 allows a surgeon to place the clip 210 at an angle to an applicator. The surgeon may also pick up the clip 210 at an angle without stressing the tissue clipped by the clip 210. As shown in FIG. 2, the applicator contact area 232 when formed as a socket, may have a side opposed to the pivot 224 formed as a raised lip 233. The raised lip or flared back surface 233 of the contact area 232 provides for extra applicator tip 272 purchase and provides a visual cue for correct placement of the applicator tips, particularly when viewed from the side.

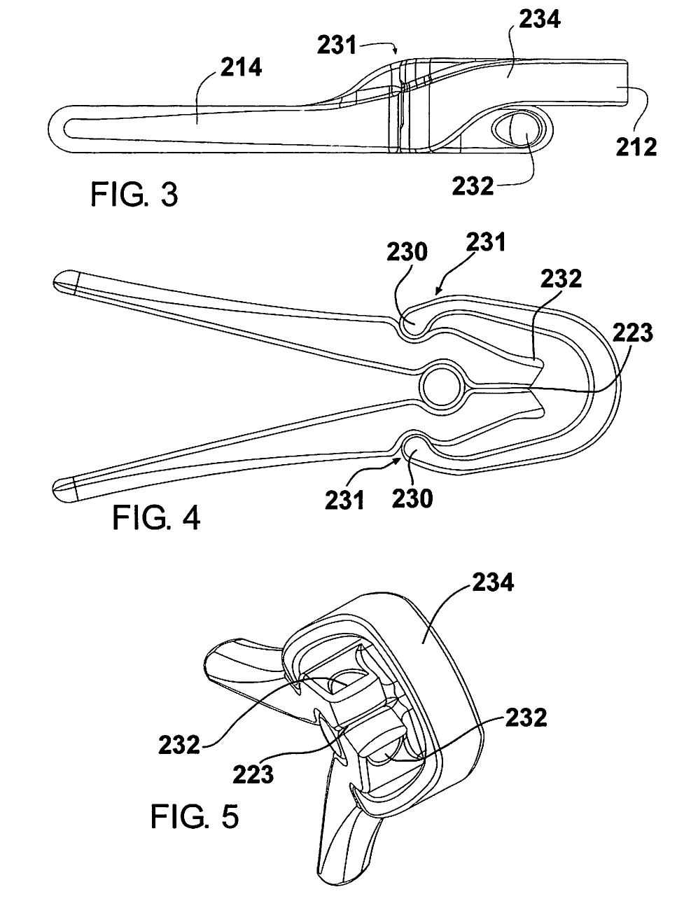

As shown in FIGS. 2 and 3, in an embodiment, the hinge joints 231 may be wider in a direction parallel to the pivot axis 226 than the jaws 214 are wide in a direction parallel to the pivot axis 226 at the clamping faces 218 of the jaws 214. Likewise, the pivot 224 may be wider along the pivot axis 226 than the jaws 214 are wide at the clamping faces 218. Other relative widths of jaw-hinge-spring may be used in other embodiments. The extra width in the spring area provides greater lateral stiffness for greater safety and precision. Using an offset spring also provides increased visibility for a surgeon. As shown in FIG. 3, the spring 212, pivot 224, spring end 228 and jaws 214 may be flush on one side, for example the side that does not have the offset portion of the spring 212.

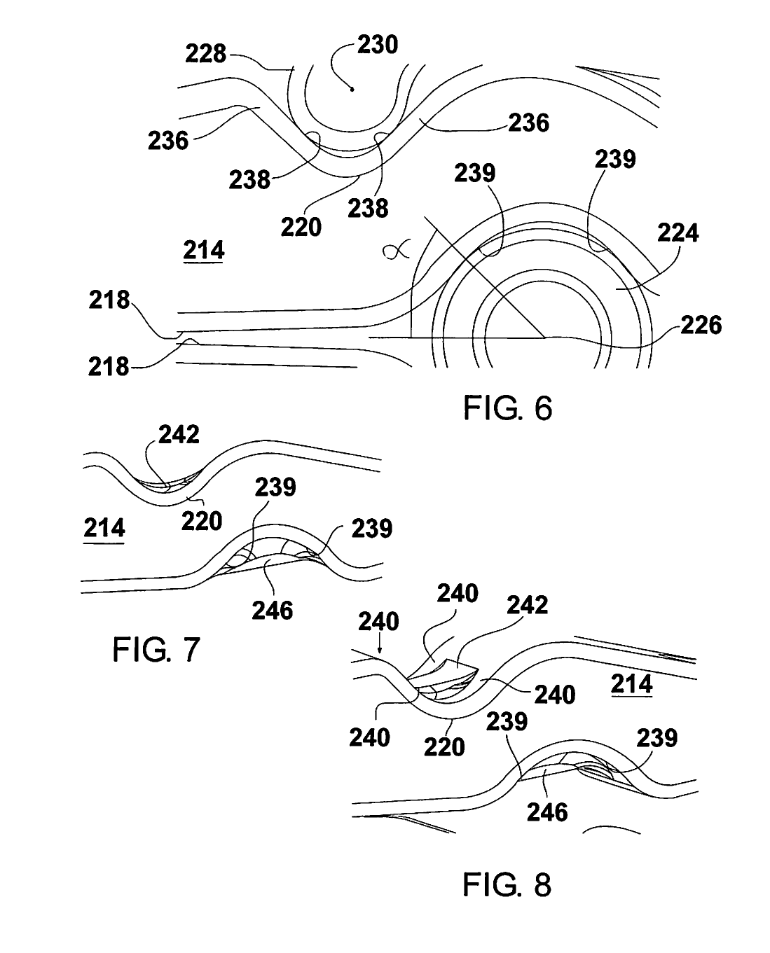

As shown in FIG. 6, each hinge joint 231 comprises confining walls 236. The confining walls 236 having sloped contact faces 238. In the embodiment shown, the sloped contact faces 238 are at an angle of 90 degrees to each other. In other embodiments, the sloped contact faces 238 may be angled at between 150 degrees and 20 degrees to each other. The smaller the angle, the greater the friction of the spring ends 228 in the sockets 220, and the larger the angle, that is, the more spread out the socket 220, the lower the stability of the joint. The sloped contact faces 238 need not have the same angle with respect to the jaw clamping faces 218, providing, in this embodiment, that the hinge joint 231 is maintained. In other embodiments, the sloped contact faces 238 of each hinge joint 231 may be angled at between 80 degrees and 100 degrees to each other. As shown in FIGS. 1-6, the confining walls 236 of each hinge joint 231 are formed as part of a respective one of the jaws 214. However, in other embodiments, the confining walls 236 may be formed in the spring ends 228, and a ridge, of for example similar shape to the spring ends 228, may be formed on each jaw 214.

Also as shown in FIG. 6, the pivot 224 may contact the jaws 214 at confining walls with sloped contact faces 239 formed in the jaws 214. The arrangement of the pivot 224 within the jaws 214 enables the pivot 224 also to provide a hinge action for the motion of the jaws 214 relative to each other. In the embodiment shown, the sloped contact faces 239 are at an angle of 90 degrees to each other. In other embodiments, the sloped contact faces 239 may be angled at between 140 degrees and 40 degrees to each other. The smaller the angle, the greater the friction of the pivot 224 against the jaws 214, and the larger the angle, the lower the stability of the pivot 224. The sloped contact faces 239 need not have the same angle with respect to the jaw clamping faces 218, providing, in this embodiment, that the pivot is maintained. In other embodiments, the sloped contact faces 239 confining the pivot 224 may be angled at between 80 degrees and 100 degrees to each other. As shown in FIGS. 1-6, the sloped contact faces 239 are formed as part of a respective one of the jaws 214. However, in other embodiments, the pivot 224 could be provided with sockets that receive protrusions from the jaws 214. In another embodiment, the pivot 224 may be formed as part of one of the jaws 214, while being received by sloped contact faces of the other of the jaws 214.

As shown in FIG. 8, the contact points 240 between the spring 212 and the jaws 214 at each hinge joint 231 may be distributed spatially in at least two dimensions, one dimension being parallel to the hinge axis 230 and another dimension being perpendicular to the hinge axis 230. In addition, as shown in FIGS. 2, 3, 5 and 8, the contact points 240 at each hinge joint may be spread parallel to the hinge axis 230 by a greater amount than the contact points 240 are spread perpendicular to the hinge axis 230. In some embodiments, the contact points 240 may contact together to form a linear contact, or may comprise four small contact areas. The sloped contact faces 238 may be flat, convex or concave, flat being relatively easier to manufacture. The sloped contact faces 238 support the hinge action of the hinge joint 231 by requiring the spring to expand if the jaws 214 are twisted. A seating arrangement with multiple distributed contact points at the spring and pivot contacts provides lateral stability. Having an arc surface very close to in contact with the spring ends and pivot at the bottom of the contact groove helps to provide a consistent and repeatable closing force for a number of cycles for each clip by providing a much greater surface area which will wear in much slower after the spring and pivot have seated themselves after the initial seating cycles.

As shown in FIGS. 7 and 8, in an embodiment of a surgical clip, the hinge joint may have a centering mechanism, such as tongue and groove structure. One of the tongue and groove is formed in the jaw 214 and the other in the spring end 228. In FIGS. 7 and 8, a tongue 242 is formed in the jaw 214. The tongue 242 is oriented perpendicular to the hinge axis 230 to oppose relative motion of the spring 212 and jaws 214 parallel to the hinge axis 230. In like manner, as shown in FIGS. 7 and 8, the sloped contact faces 239 confining the pivot 224 in the jaws 214 may also have contact areas 244 with a like distribution to the contact points 240, and have centering structures such as the tongue or web 246, with a corresponding feature in the pivot 224. That is, either the jaws 214 or the pivot 224 may have the tongue, and the other the groove.

As shown in FIGS. 1 and 6, in one embodiment, the hinge joints 231 are located with respect to the pivot 224 so that lines joining axes 230 of each hinge joint 231 to the pivot axis 226 form an angle 2α of greater than 90 degrees, as for example 104 degrees. In one embodiment, the hinge axes 230 are symmetrically arranged in relation to a plane bisecting the jaws 214 and passing through the pivot axis 226. A high angle α tends to position the spring 212 further from the jaws 214, which makes imaging of a body part clipped by the jaws 214 easier. A high angle α also requires a shorter spring 212 due to the decreased spring deflection for a given jaw opening angle. A higher angle α also requires a greater spring force which provides increased ability to the assembly.

In one embodiment, as shown in FIGS. 9 and 10, the contact points 240, 246 at each hinge joint 231 and pivot 224 respectively are distributed about a gap 248, 250 between the spring ends 228 and respective jaw 214 on the one hand, and between the jaws 214 and the pivot 224 on the other hand. In each case, the gap 248, 250 extends parallel to the hinge axis 230 or pivot axis 226 as the case may be. The gap 248, 250 allows initial contact of the parts to be angular, with gradual wear or contact pressure causing a greater contact area, and subsequent reduction in wear. Initial contact forces are quite high, so the spring ends 228 or the pivot 224 can reduce in size in only a few cycles of compression perhaps as low as 2-10 cycles in some configurations before the gaps 248, 250 are eliminated and the spring ends 228 or pivot 224 bottom out. Gaps 248, 250 are preferably 0.0005 inches but may be in the order of 0.0001 inches to 0.005 inches for a surgical clip having size suitable for use as an aneurysm clip. The sum of the thicknesses of the four gaps, two gaps between the hinges and the jaws and two between the pivot and jaws, may in one embodiment be less than the initial pre-load on the spring 212 when the jaws 214 are closed. The initial pre-load is the total deflection of the spring ends 228 between the resting position (spring is not loaded) and the position of the spring ends 228 when the jaws 214 are closed. These gaps provided increased closing force consistency over a number of cycles. As the initial contact points wear, the spring ends 228 comes into contact with the bottom of the sockets 220 and greatly increase the surface contact area between the spring ends 228 and sockets 220. Thus, the gaps tend to minimize wear while retaining lateral stability of the hinge joints 231 and pivot 224.

The jaws 214, particularly for use as an aneurysm clip, may be made at least in part of a strong MRI invisible material such as ceramic, as for example zirconia, alumina, silicon carbide or silicon nitride to facilitate MR imaging during or immediately after placement of the clip or long term periodic imaging to monitor the aneurysm after surgery, or may be made of other MRI invisible bio-compatible material or mixtures thereof such as PEEK (Polyetheretherketone), PEI PA (Polyamide—nylons PA-6, PA-12, PA-66), PEEK composite, where the PEEK is reinforced with fibers such as carbon fiber, aramid, graphite or glass fiber and glass of various types such as heat strengthened glass, ion-exchange surface-compression strengthened (SCS) glass or borosilicate glass (such as Pyrex™ brand). By strong is meant sufficiently strong to clamp on and occlude an aneurysm or vessel without breaking normal operation. A material is MRI invisible when it has a weak artifact in MR imaging that does not substantially block or distort the MR image. Materials which provide MR imaging benefits generally provide benefits for x-ray imaging as well.

The spring 212 and pivot 224 may be made of an MRI compatible bio-compatible material such as titanium or titanium alloy or cobalt alloy. Exemplary titanium alloys are Ti-6Al-4V titanium alloys, or other biocompatible materials under ASTM F-136 (ISO 5832-3). In some embodiments, the jaws 214 may also be made of titanium or titanium alloy. Combinations of materials may also be used, so that for example the pivot or jaws may be part metal alloy and part ceramic in some embodiments of a surgical clip. MRI compatible means that the material is essentially non-magnetic, non-electrically conducting and non RF reactive.

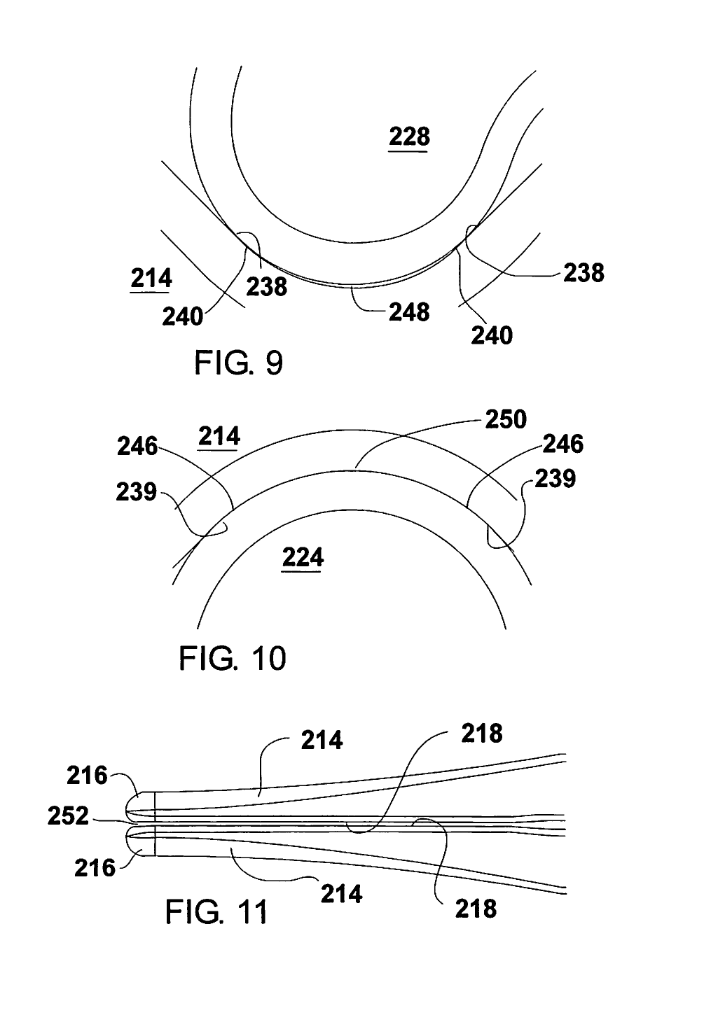

The thickness of the pivot 224 may be used to control spacing of the jaws 214. In FIG. 11, a clip 210 shown in a partly open position. The jaws 214 are spaced by the pivot 224 (not shown in FIG. 11) so that the clamping faces 218 are only parallel when the jaws 214 are slightly open, In the slightly open, parallel position, the clamping faces 218 are spaced from each other with a gap 252. For a typical size of aneurysm clip, the gap 252 may be about 0.004 inches. In this embodiment, when the jaws 214 are clamped together by the spring 212, the clamping ends 216 contact before the remainder of the clamping faces 218. This design helps prevent high clamping forces being generated near the pivot 224.

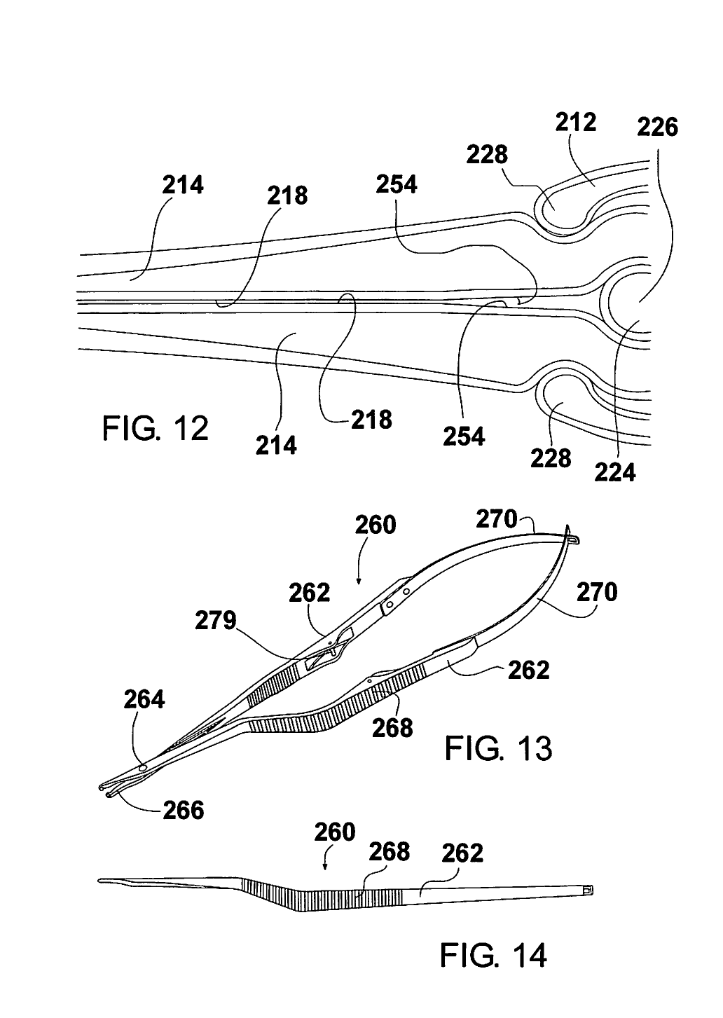

As shown in FIG. 12, in an embodiment, the clamping faces 218 may each have a divergent portion 254, so that, when at least a portion of the clamping faces 218 of the jaws 214 are parallel, the divergent portions 254 diverge away from each other, the divergence being the greater the closer to the pivot 224. Also, as shown in the embodiment of FIGS. 11 and 12, the jaws 214 may taper or thin towards the clamping ends 216 when measured in a direction perpendicular to the pivot axis 226. A thinner profile at the clamping ends 216 facilitates placement of the clip in a desired position, while the thicker portion of the jaws 214 retains the benefits of extra strength, including allowing use of weaker materials. The ramping nature of the outer surface of the jaws 214 also assists in deflecting tissue away from the spring ends 228, and helps prevent the spring ends 228 catching on tissue.

A variety of surgical clip designs are described and claimed in this patent document. In one design, the joint connecting a spring to jaws of a clip is a hinge joint. In certain designs, the joint connecting a spring to jaws of a clip need not be a hinge joint, such as in designs that include any one or more of the following features: the jaws being formed of ceramic and the spring being formed of titanium or titanium alloy; each applicator end comprising a ball or socket; the pivot being wider in a direction parallel to the pivot axis than the jaws are wide at the clamping faces of the jaws; the pivot separating the jaws so that, when at least a portion of the clamping faces of the jaws are parallel, the clamping faces are spaced from each other; the jaws tapering towards the clamping ends when measured in a direction perpendicular to the pivot axis; the jaws each separating from each other to form respective arches in a portion between the clamping faces and the pivot; the clamping faces each having a long dimension and a short dimension, and the long dimension being parallel to the pivot axis, for example when the jaws form an L or T shape; one of the jaws being shorter than the other jaw; or the spring incorporating at least one flange extending outward from the spring between the spring ends.

The surgical clips described above and below are manipulated by a surgeon with a medical applicator. An embodiment of a medical applicator is shown in FIGS. 13-17, in which a medical applicator 260 has a pair of arms 262 connected by a pivot 264 having a pivot axis 265, each arm of the pair of arms having a tip end 266 on one side of the pivot 264 and a handle 268 on the other side of the pivot 264. A spring formed of spring parts 270 is provided between the arms 262 to urge the arms 262 when held by a surgeon. If a single spring is used, it may be attached to one of the arms 262, but as shown each spring part 270 is attached to a different one of the arms 262. Each tip end 266 has a clip contact area 272. The handles 268 are offset from the tip ends 266, so that planes perpendicular to the pivot axis 265 and passing through the clip contact areas 272 do not pass through the handles 268. The offset or asymmetric tip design allows one side of the applicator 260, at least a portion of the side of the applicator for example up to the pivot, to line up flush with at least a portion of one side of a clip such as clip 210, such as a side of the jaws at the pivot and rearward of the pivot. The offset of the handles 268 also allows the surgeon an unobstructed view of the clip when viewing the clip from directly behind.

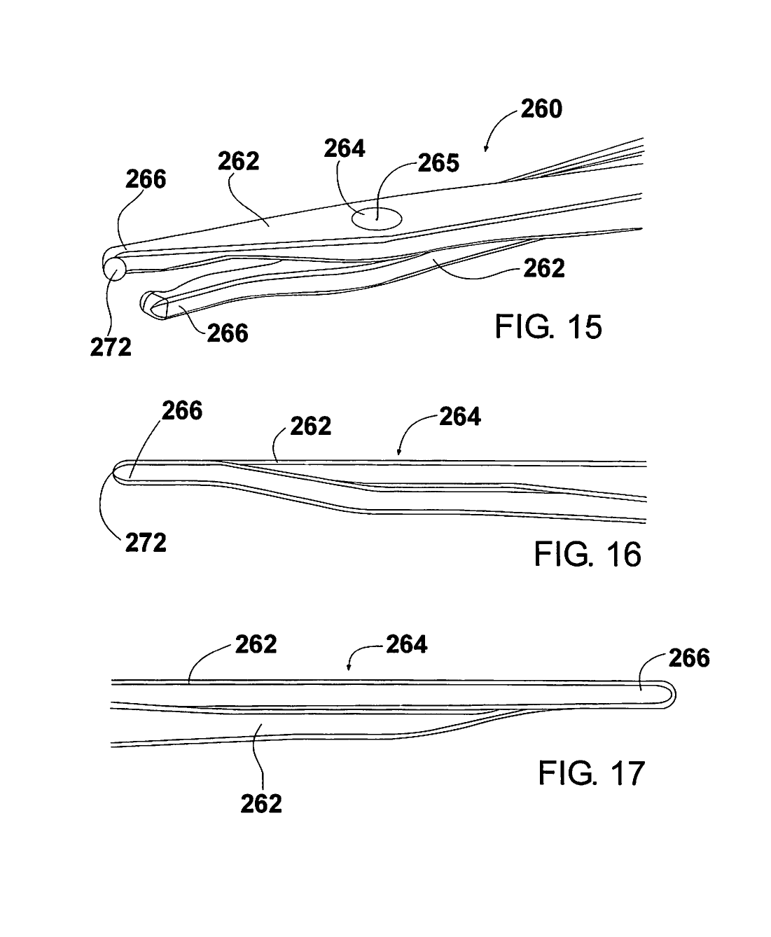

The clip contact areas 272 may in an embodiment be formed as a ball or socket as shown in FIG. 15 to mate with a respective ball or socket on a clip to be manipulated by the medical applicator. The ball and socket each incorporate matching spherical surfaces. As shown, the ball is on the applicator of FIGS. 13-17, and the socket is on the surgical clip of FIGS. 1-12. In an embodiment, the balls could be on the surgical clip, and the sockets on the applicator. In another embodiment, one applicator contact area on the clip may be a ball and the other a socket, and likewise for the clip contact area on the applicator.

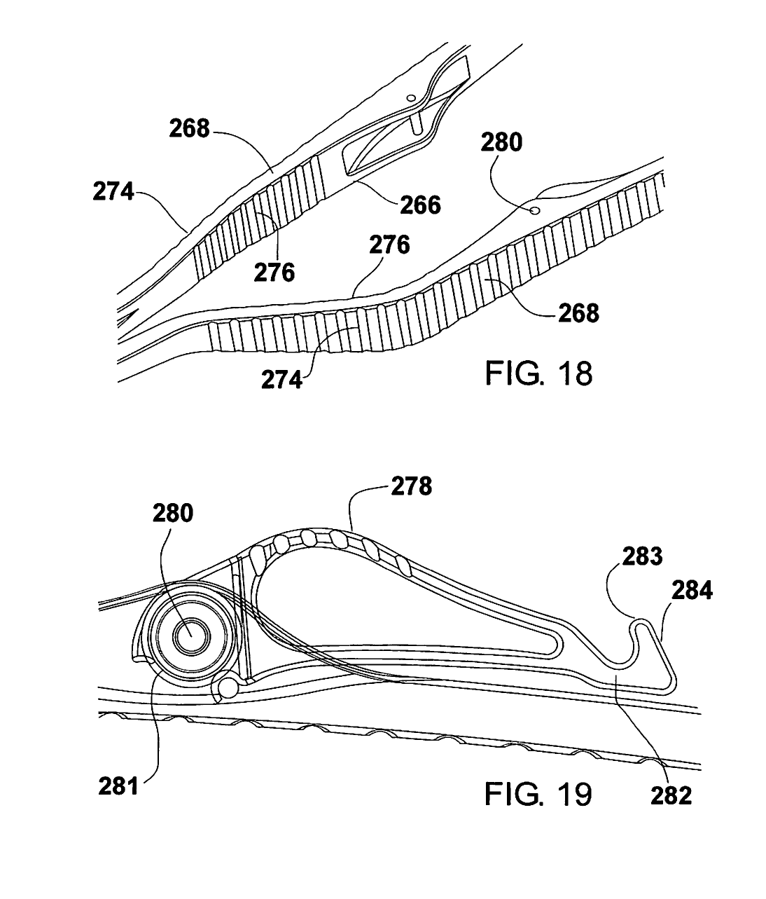

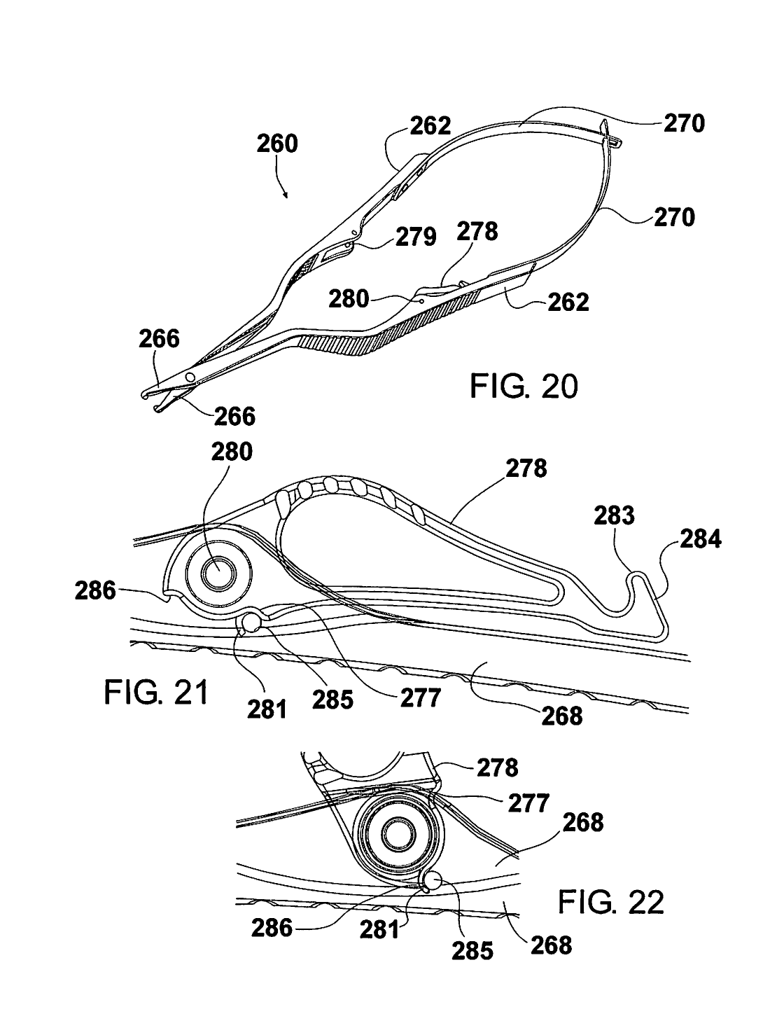

As shown in FIGS. 13 and 14, the handles 268 have exterior grip surfaces 274, and also as shown in FIG. 18 may have grip surfaces 276 on inner portions of the handles 268. A latch 278 may be provided on the medical application 260 as shown in FIGS. 19-24. In the embodiment of FIGS. 19-24, the medical applicator 260 has the latch 278 secured to one handle 268 by a latch pivot 280. A catch 279 is provided on the other handle 80. The latch pivot 280 may extend fully across the handle 80 to which it is attached to resist bending of the latch 278. Bending resistance both keeps the latch 278 functional and assists in preventing a surgeon deliberately moving the latch 278 out of the way. The latch 278 is spring mounted on the pivot 280 with a spring 281, shown in the cut-away of FIG. 19, that biases the latch 278 towards the handle 268 to which it is attached. The latch bias provides the dual function of allowing the surgeon to control the application of the latch, and also keeps the latch 278 conveniently out of the way when not in use as shown in FIG. 21.

The latch 278 has a latching end 282 with a hook 283 having a slanted inwardly widening exterior face 284. A stop structure, for example incorporating a pin 285, is provided adjacent the pivot 280. The pin 285 prevents contact of the latching end 282 of the latch 278 with the handle 268 to which it is attached, and provides a solid abutment where the pin 285 contacts the latch 278 at shoulder 277. By contact with shoulder 286 on the latch 278, the pin 285 also functions as a stop to prevent rotation of the latch 278 by more than a pre-defined distance beyond the catch 279 and to allow the face 284 of the hook 283 to contact the catch 279 when the latch 278 is deployed. The two stop functions provided by the pin 285 may be provided by two abutments such as two pins. As shown in FIG. 22, the pin 285 may also function as an anchor for the spring 281.

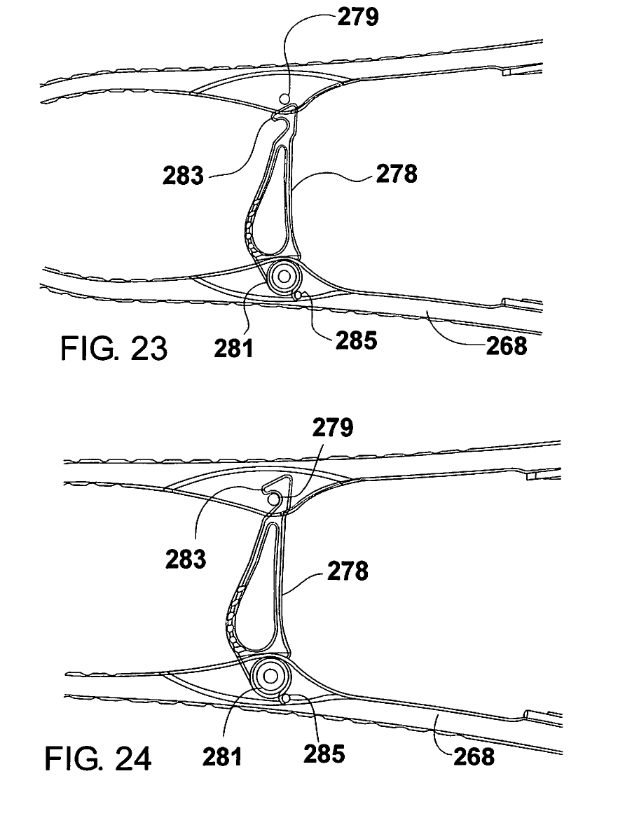

The latch 278 is engaged with the catch 279 by the surgeon moving the latch 278 from the stowed position of FIG. 21 until the shoulder 286 engages the pin 285 as shown in FIGS. 22 and 23. When the latch 278 engages the second stop 286, the exterior face 284 of the hook 283 contacts or nearly contacts the catch 279. A slight closing of the handles 268 in this position with pressure exerted by the surgeon on the latch 278 towards the catch 279 causes the hook 283 to engage the catch 279 as shown in FIG. 24. To release the latch, the surgeon applies a slight closing pressure to the applicator handles 268. The slight closing in the case of an applicator used with an aneurysm clip may be in the order of 0.01 inches, but may be more or less. Upon release of the latch 278 by a slight closing of the handles 268, the latch 278 swings towards the handle 268 to which it is attached. Upon contact of the latch 278 with the handle 268, the base of the latch 278 close to the pivot 280 stops abruptly and the continuing momentum of the distal portions of the latch 278 provides tactile feedback for the surgeon to detect that the latch to 78 has been stowed out of the way.

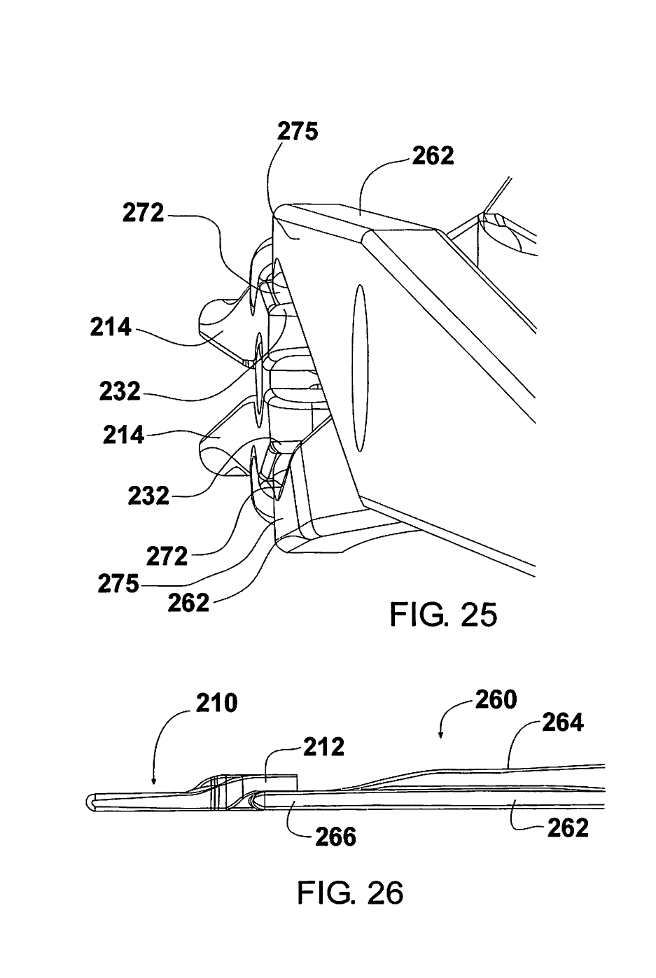

As shown in FIGS. 25 and 26, for an embodiment of an applicator 260 used with a clip 210, the clip 210 is slightly open when the latch 278 is engaged. This provides a secure grip on the clip 210 and gives the user a visual indication that the latch 278 is engaged. Preferably, the clip 210 is not more than half open, to avoid the surgeon having to open the latch 278 if the clip 210 is too wide and needs to be closed more. As shown in FIGS. 25 and 26, the clip 210 surfaces on the side opposed to the spring 212 are flush with the applicator surfaces 275 on the side of the applicator away from the handles 268.

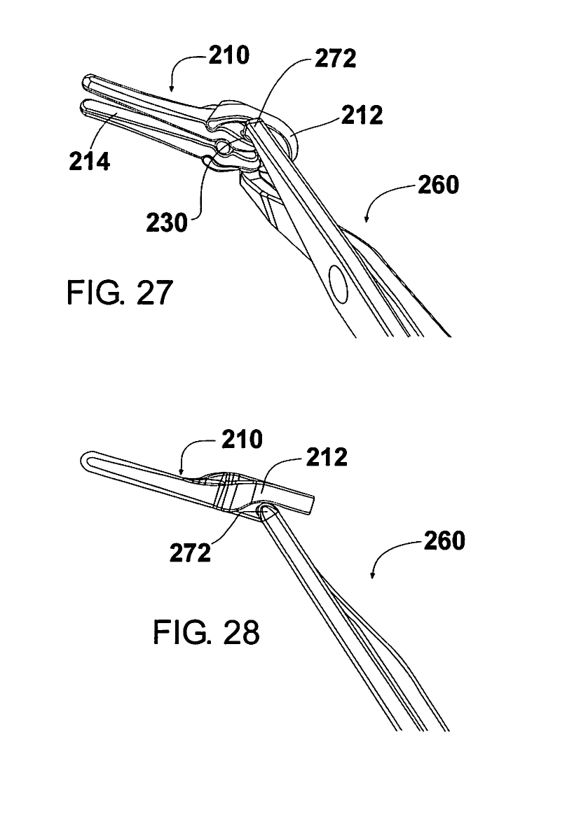

The ball and socket configuration of the clip-applicator contact areas allows applicator gripping over a variety of angles, as shown in FIGS. 27 and 28. The spring 212 confines the movement to one side only. Angular placement of the applicator tips 266 on the clip 210 allows the surgeon to use a straight clip 210 in a wide range of placements. In addition, the ball and socket arrangement allows the contact areas 232, 272 to wear without losing their function. The applicator 260 may for example be made of titanium alloy or other bio-compatible and preferably MRI compatible material and when the applicator contact areas 232 are made of ceramic or other material of like hardness, the ceramic tends to shape the titanium alloy through use. The spring 281 may also be made of titanium alloy or other suitable biomedical material, and the pivot 280 may for example be made of titanium alloy or ceramic or both.

The applicator of FIGS. 13-27 works by an operator closing the handles 268 against force provided by the applicator spring 270. The operator then moves the latch 278 against force provided by the latch spring 281 until the hook 283 abuts against the catch 279. The operator then continues to close the handles 268 together while pressing the hook 283 against the catch 279 until the hook 283 latches onto the catch 279.

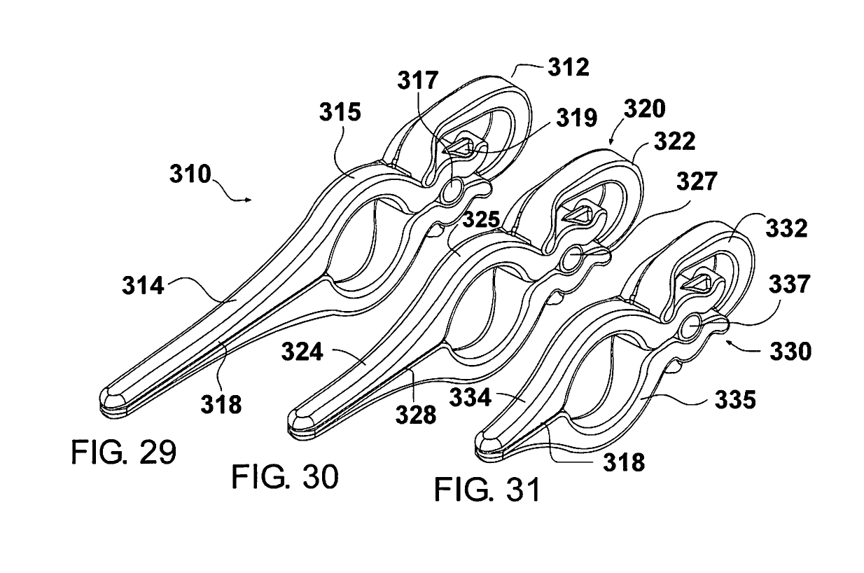

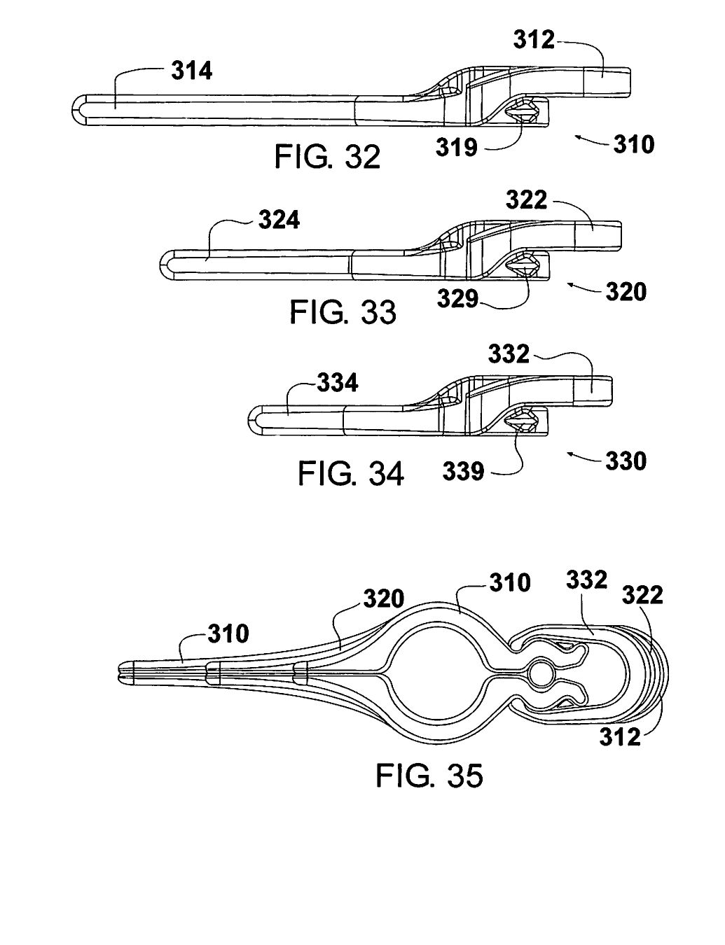

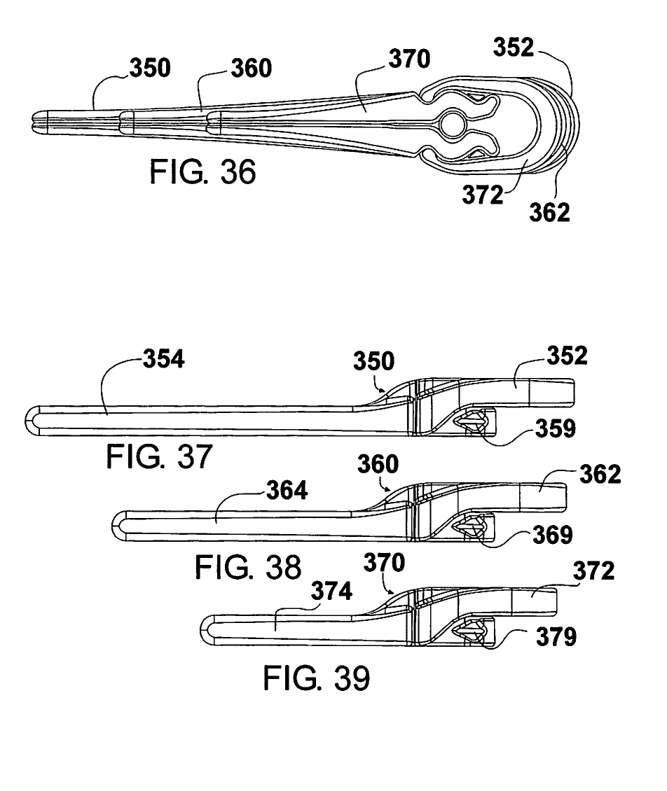

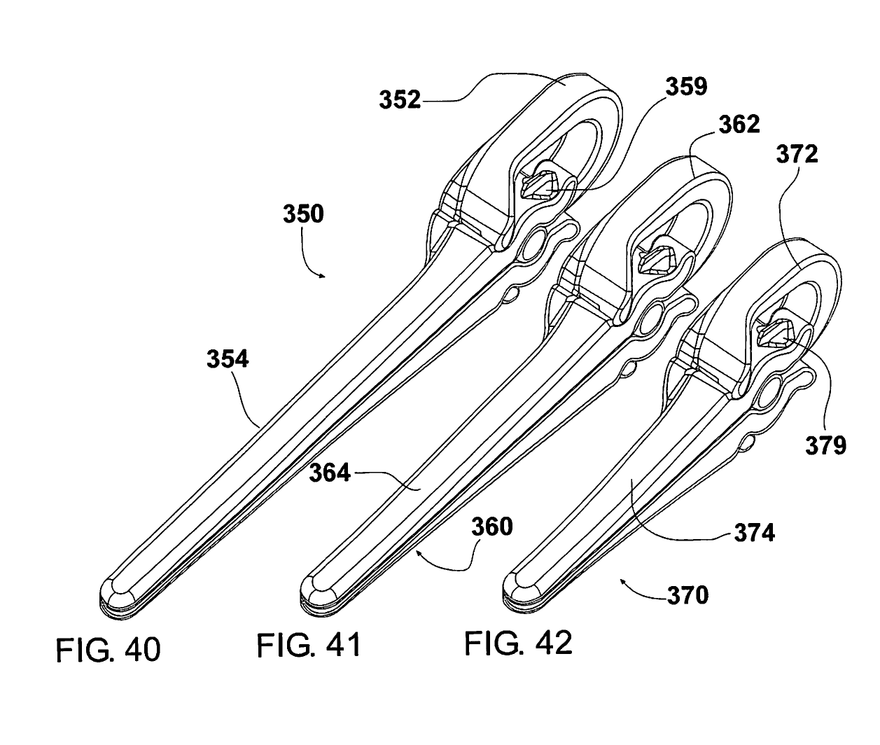

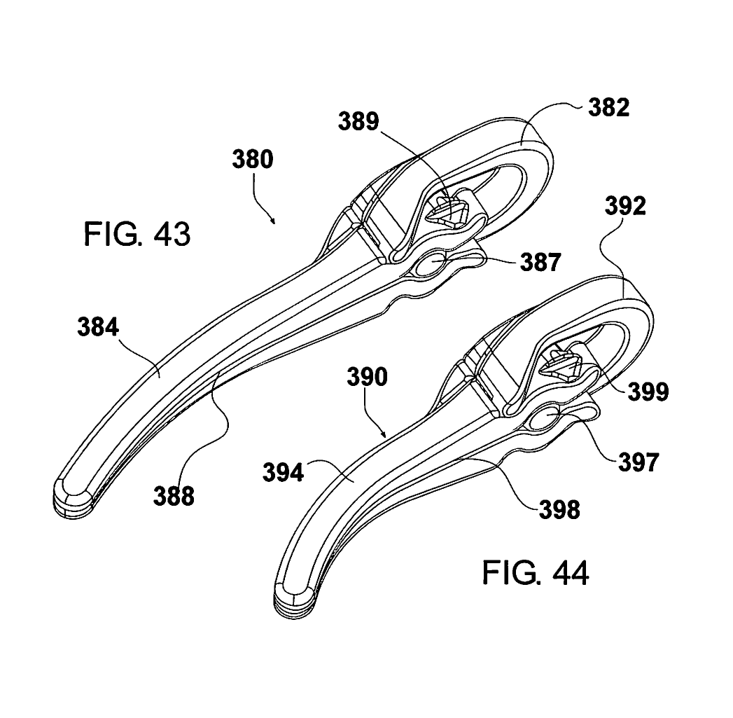

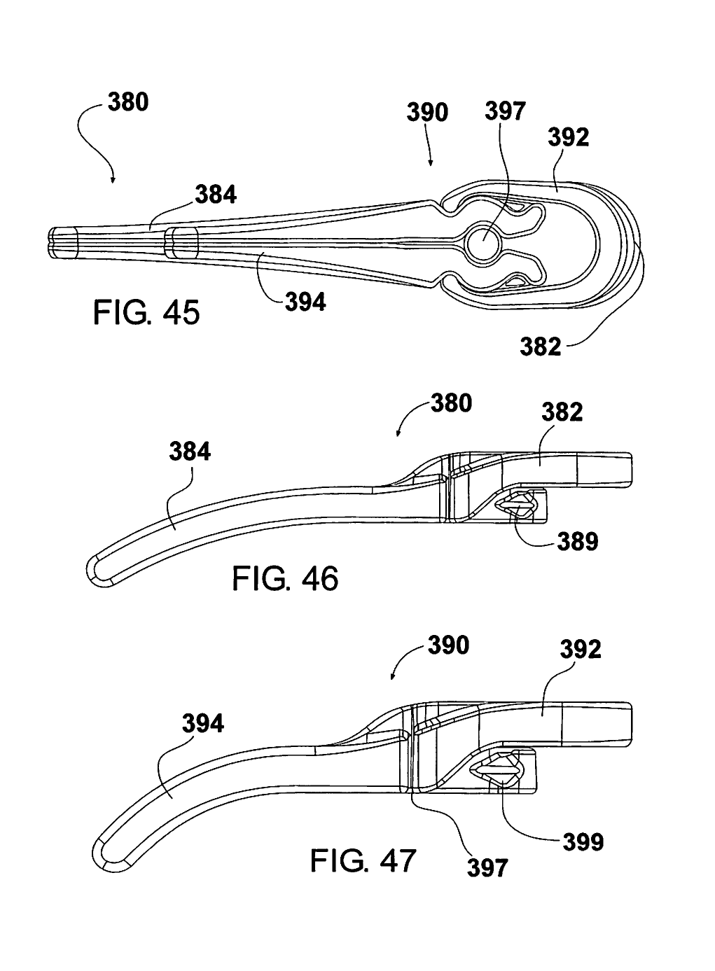

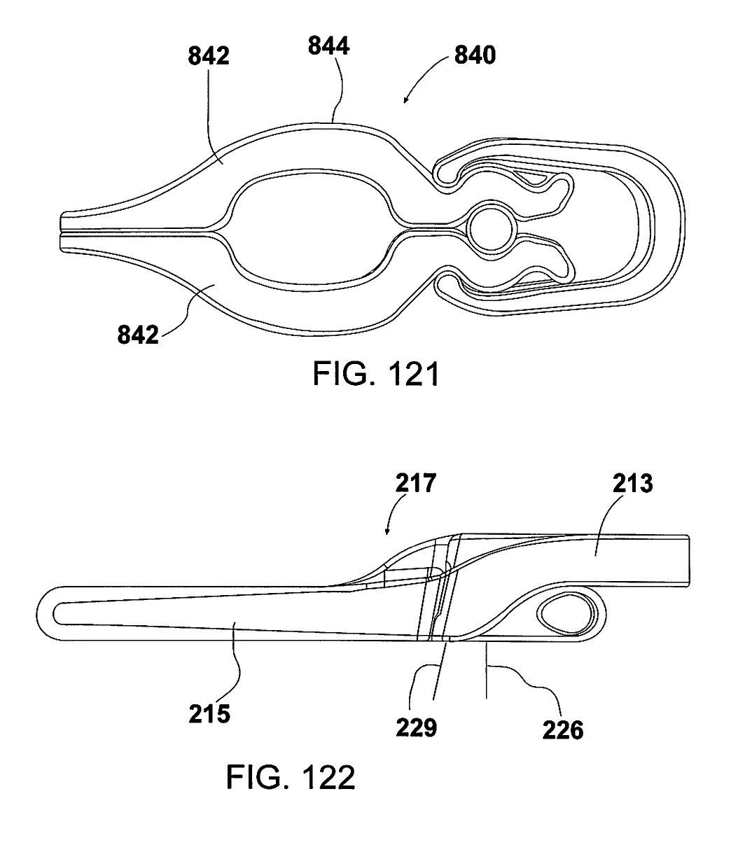

As shown in FIGS. 29-35, embodiments 310, 320 and 330 of a surgical clip may have jaws 314, 324 and 334 respectively that each separate or diverge from each other to form respective arches 315, 325 and 335 in a portion of the jaws between the clamping faces 318, 328 and 338 respectively and the pivots 317, 327 and 337. The embodiments 310, 320 and 330 show various sizes of surgical clips, with springs 312, 322 and 332 that may be designed according to the design of spring 212. The respective arches 315, 325 and 335 combine to form a circular jaw portion. FIGS. 36-42 show various sizes of surgical clips 350, 360 and 370, with straight jaws 354, 364 and 374 and springs 352, 362 and 372. FIGS. 43-47 show embodiments of surgical clips 380 and 390, with respective springs 382 and 392, and jaws 384 and 394 that curve in a plane parallel to the gap between the clamping faces 388, 398 that passes through the pivot axes 387 and 397. As shown in FIG. 121, a clip 840 in one embodiment may have divergent jaws 842 forming a polygonal arch 844. The polygonal arch 844 may be regular (equal length sides) or irregular. As with the circular arch, the polygonal arch may be formed on one or both jaws.

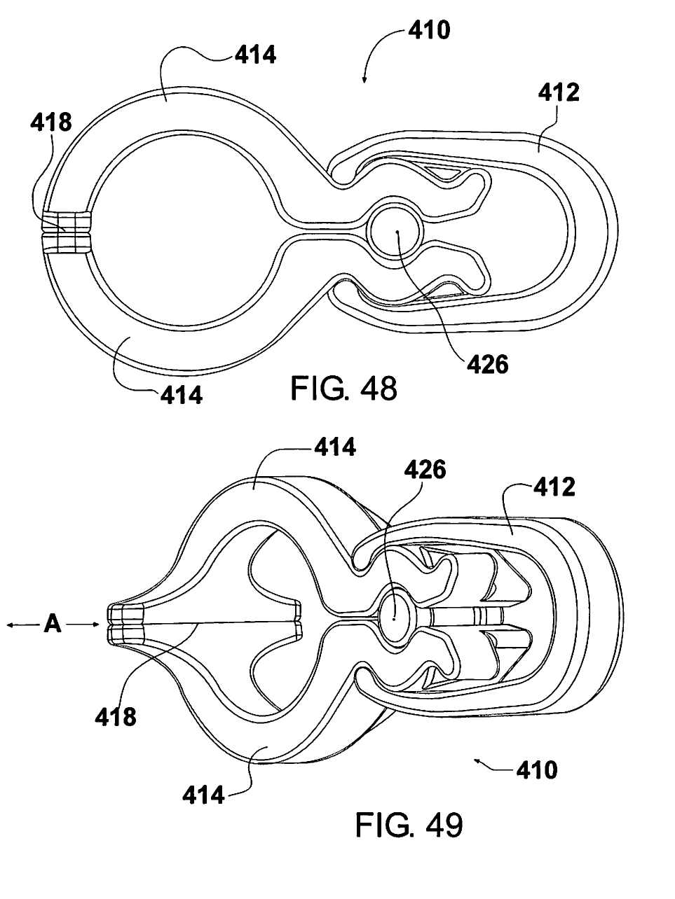

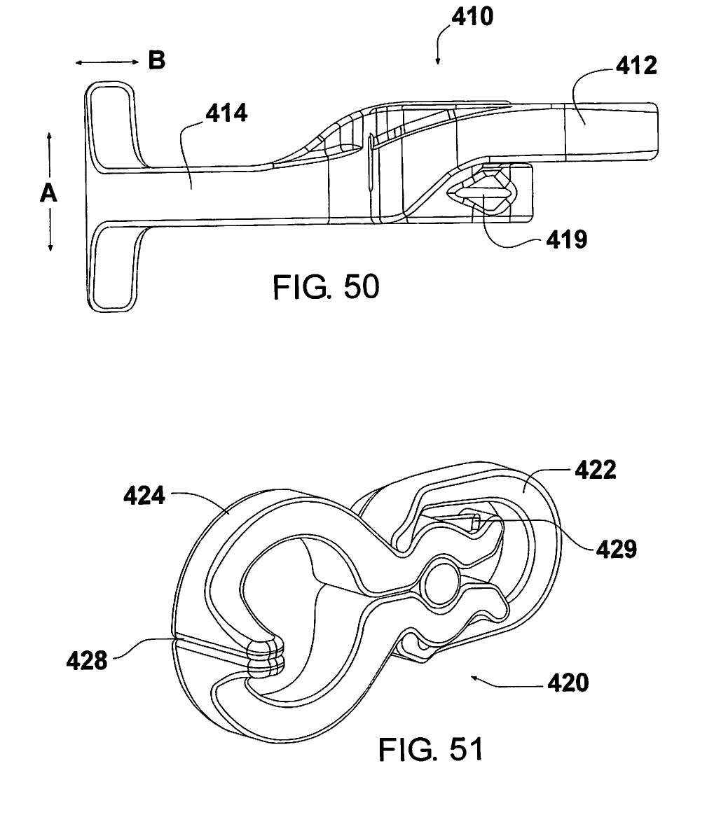

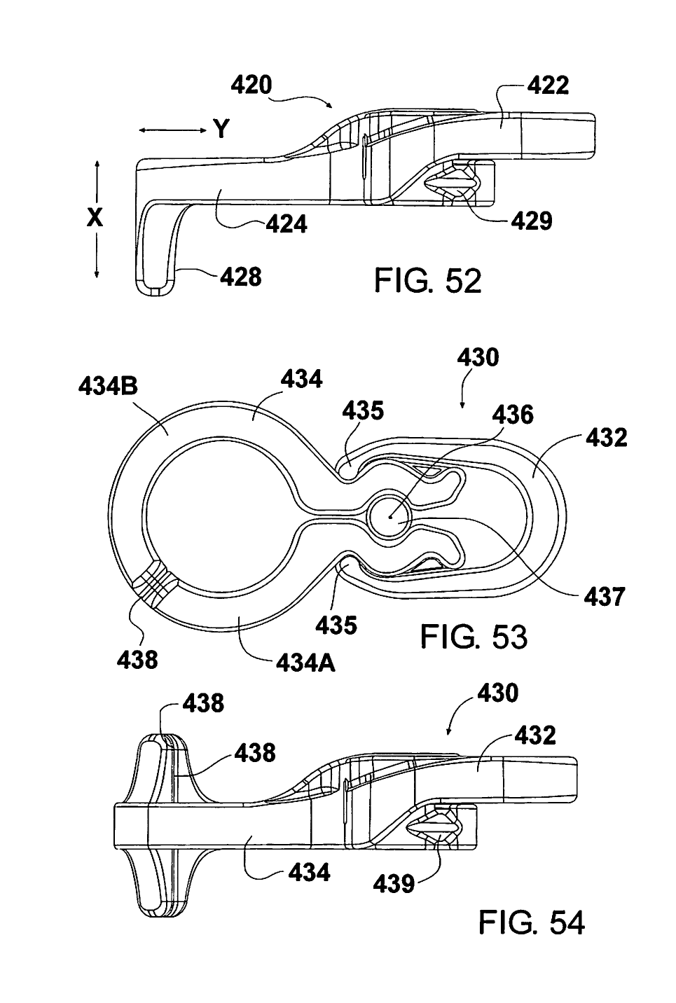

Referring to FIGS. 48-50, an embodiment 410 of a surgical clip is shown, with jaws 414 and spring 412, in which the clamping faces 418 each have a long dimension A and a short dimension B, and the long dimension is parallel to the pivot axis 426. The jaws 414 form a T shape, as shown in FIGS. 49 and 50, with, in the embodiment shown, the jaws 414 inward of the clamping faces 418 forming a circular portion. In FIGS. 51-52, a surgical clip 420 is shown, with jaws 424 and spring 422, in which the clamping faces 428 each have a long dimension X and short dimension Y, and the clamping faces 428 extend laterally only on one side of the jaws 424 to form an L-shape with the jaws. In these embodiments of FIGS. 48-52, the clamping faces 418 and 428 are formed as part of an arm or arms of the L or T shape. IN some embodiments of a clip with T-shaped jaws, the jaws do not have a circular portion, but may be straight or curved only in a plane parallel to the clamping faces. The arms of the T may also be at an angle to the main part of the jaws, or the arms may be curved in the plane of the clamping faces, curving towards or away from the spring end.

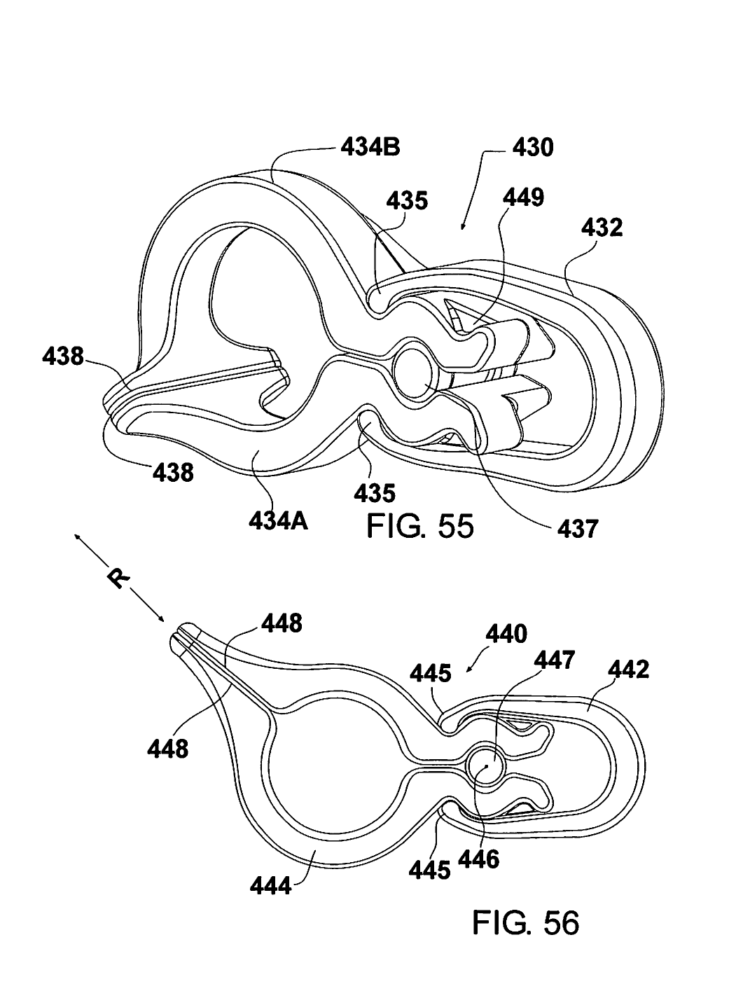

In an embodiment of a surgical clip shown in FIGS. 53-55, a surgical clip 430 has circular jaws 434, a spring 432, clamping faces 438, spring ends 435, pivot 437 and pivot axis 436. The jaws 434 are of different lengths, so that jaw 434A is shorter than jaw 434B. The clamping faces 438 thus form an angle of between zero degrees and 90 degrees to a plane that passes through the pivot axis 436 and that passes half way between the spring ends 435. In the embodiment shown in FIGS. 53-55, the angle is 45 degrees.

In the embodiment of FIGS. 56-57, a surgical clip 440 has circular jaws 444, a spring 442, clamping faces 448, spring ends 445, pivot 447 and pivot axis 446. The clamping faces 448 have a long dimension R and a short dimension S and the long dimension R extends radially in relation to the circular jaw portion. The clamping faces 448 thus form an angle of between zero degrees and 90 degrees to a plane that passes through the pivot axis 446 and that passes half way between the spring ends 445. In the embodiment shown in FIGS. 56-57, the angle is 45 degrees.

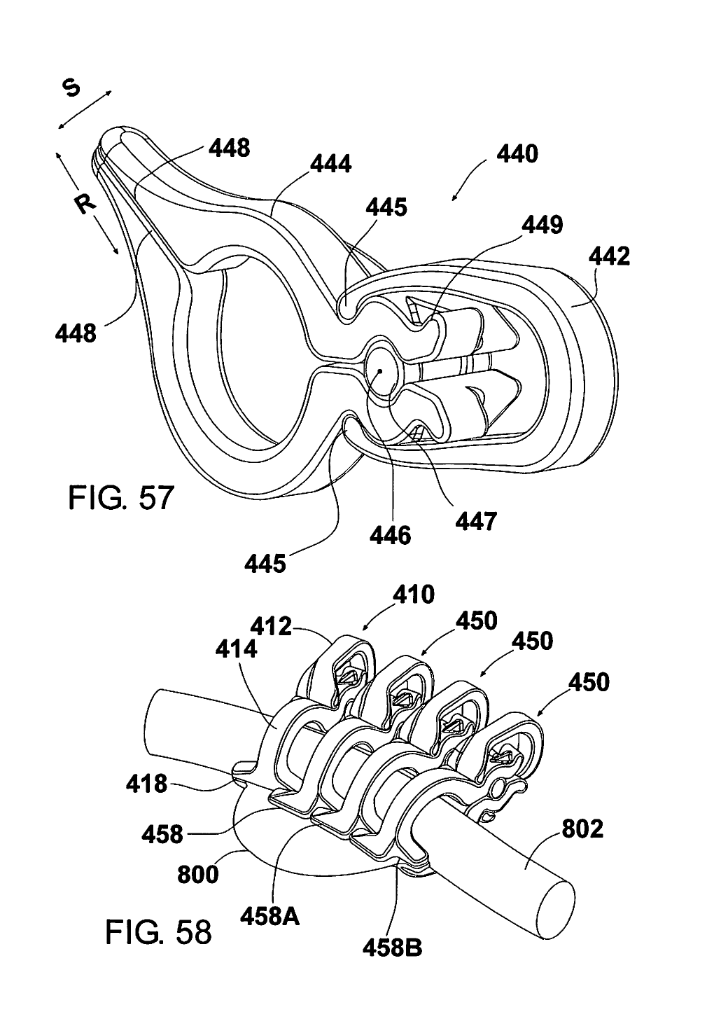

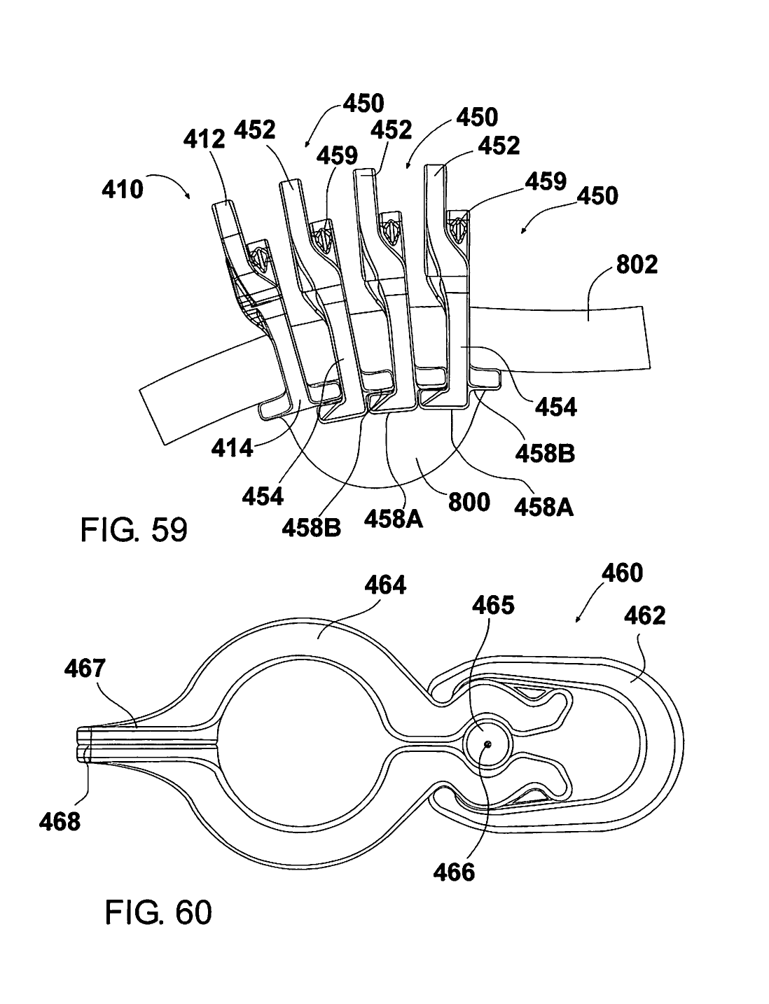

An embodiment of a surgical clip is shown in FIGS. 58-59 in which a surgical clip 450 has circular jaws 454, a spring 452, clamping faces 458A, 458B, spring ends 455, pivot 457 and pivot axis 456. In FIGS. 58-59, the clamping faces 458A, 458B of the jaws 454 form arms of the T shape, and the clamping faces 458A, 458B are offset from each other in a direction perpendicular to the pivot axis 456. The design shown in FIGS. 58-59, in which the clamping faces 458A, 458B are longitudinally offset, allows the surgical clip 450 to be nested as shown in FIGS. 58-59 so that the clamping face 458A of one surgical clip 450 overlaps the clamping face 458B of an adjacent surgical clip 450 of like construction, thus forming a stitch clip. In one embodiment, the longitudinal offset of face 458A from face 458B is equal to the width of clamping face 458B. When the faces 458A, 458B are offset from each other in this manner, and overlapped as shown in FIGS. 58-59, the clips 450 may be used to clip a larger amount of tissue, here shown as an aneurysm 800 on a vessel 802. Varying amounts of offset and clamping face width may be used to obtain varying coverage when the surgical clips 450 are nested together. The clips 450 may be used for example in conjunction with a surgical clip having the design of clip 410 as shown.

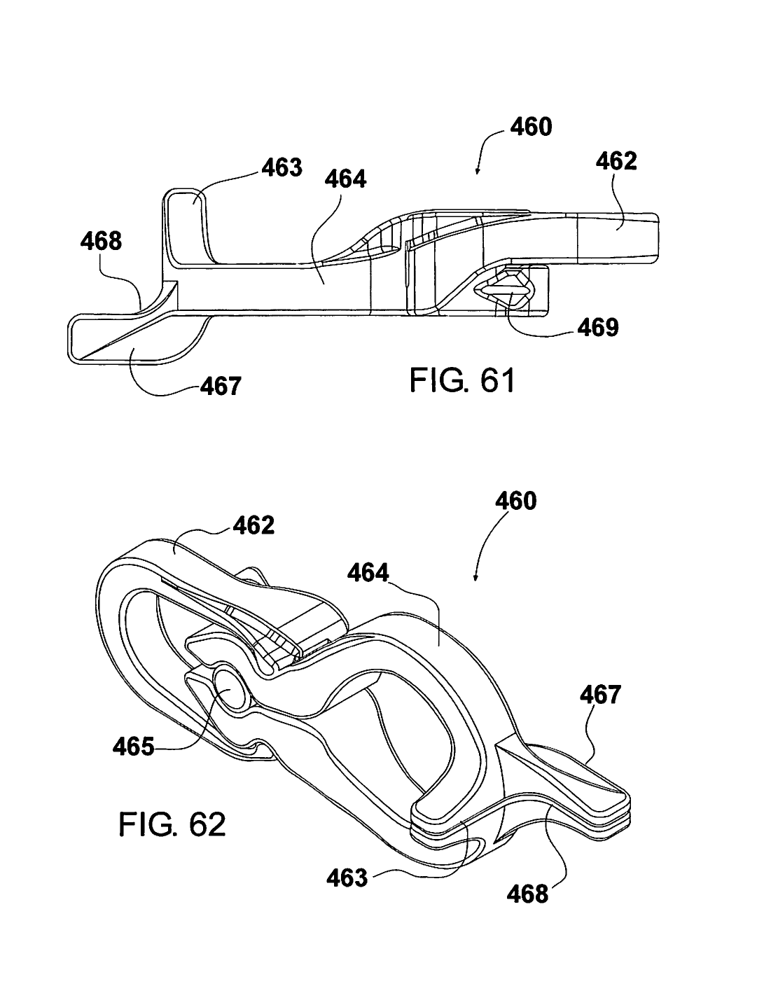

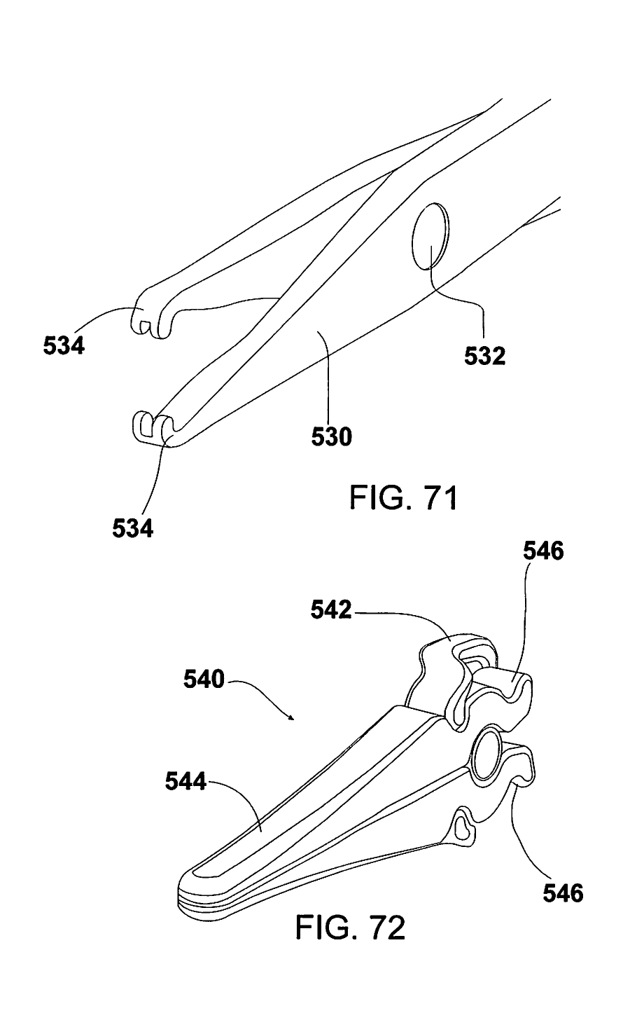

In the embodiments of clips shown in FIGS. 31-62, the various clips have respective applicator ends 319, 329, 339, 359, 369, 379, 389, 399, 419, 429, 439, 449, 459 and 469 that are designed to be operated by an applicator as shown for example in FIG. 71, but any of various applicator ends may be used in these embodiments, as for example a ball or socket applicator end.

As shown in FIGS. 60-62, an embodiment of a surgical clip 460 has jaws 464 with a circular portion, a spring 462, pivot 465, pivot axis 466 and the jaws 464 having L-shaped clamping faces 468, where one side 463 of the L-shape of the clamping faces 468 is formed as a perpendicular extension from the jaws 464, and the other side 467 is formed as an offset longitudinal extension of the jaws 464.

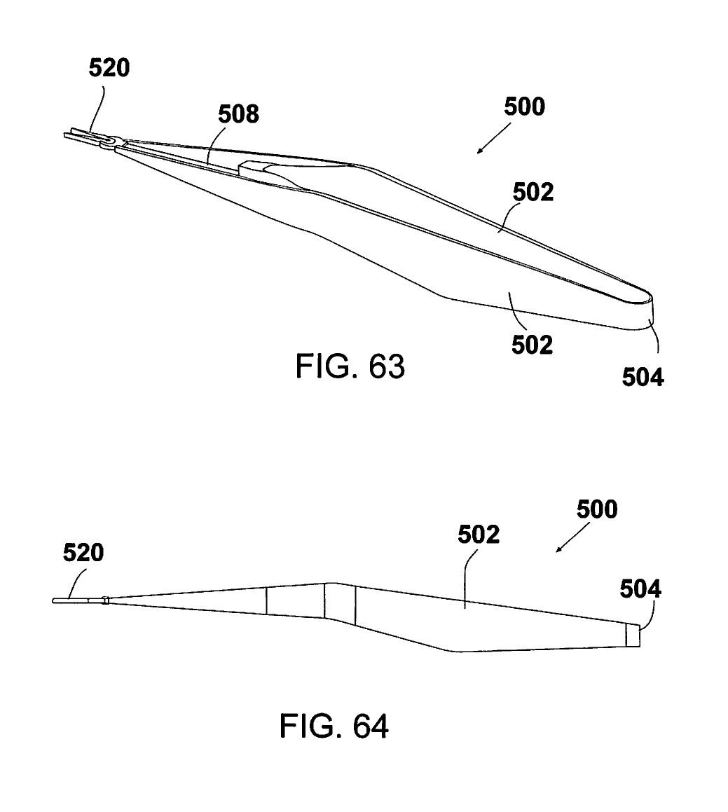

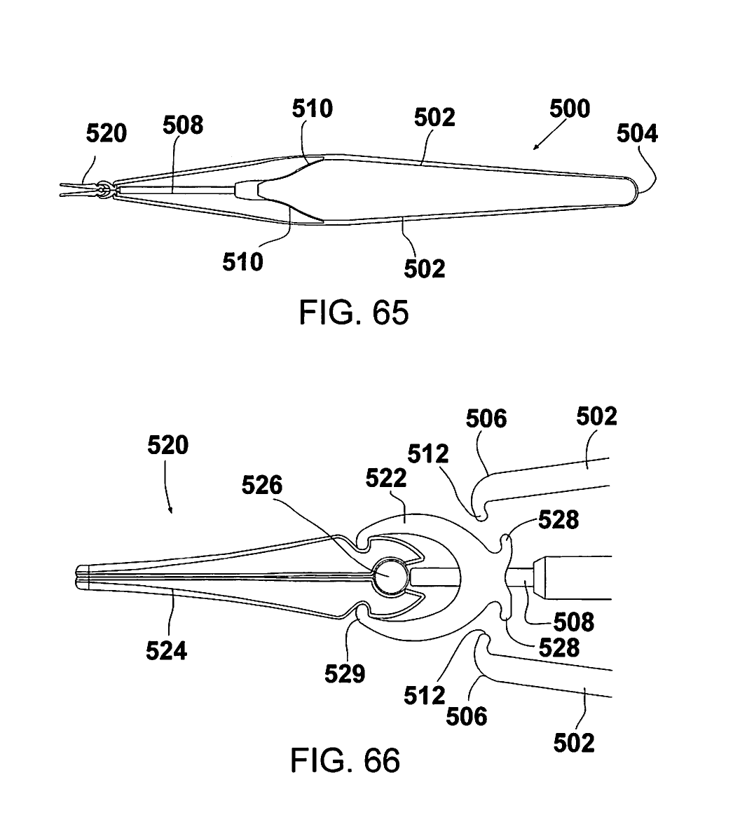

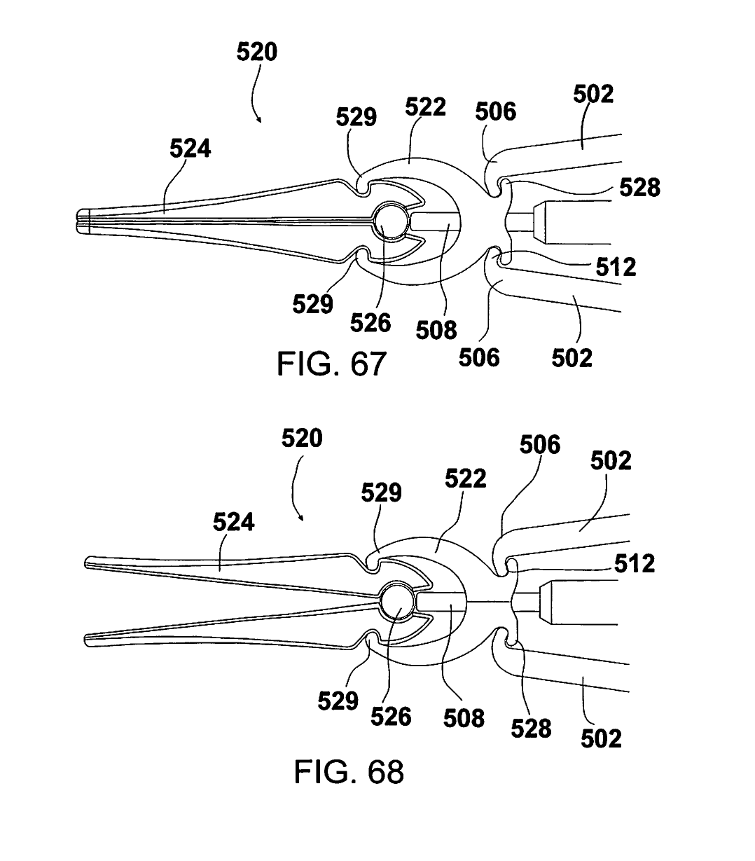

An embodiment of a medical applicator 500 is shown in FIGS. 63-70 that may be used in association with a clip 520. The medical applicator 500 has handles 502 connected to each other at an applicator pivot 504. The pivot 504 may be configured as a resilient part of the handles 502. Each handle 502 has a hook 506 at a portion of the respective handle 502 spaced from the applicator pivot 504. A pin 508 extends away from the applicator pivot 504, and the pin 508 is attached to each handle 502 by respective lever arms or leaf springs 510. The lever arms 510 are pivotally attached to each of one end of the pin 508 and to the handles 502 between the respective hooks 506 and the applicator pivot 504. The pin 508 extends further away from the applicator pivot 504 than the handles 502 extend away from the applicator pivot 504. The hooks 506 have inwardly extending hook arms 512. The applicator 500 may be formed of a single piece of titanium alloy or other suitable biomedical material, and may also be formed of individual parts made of biomedical materials.

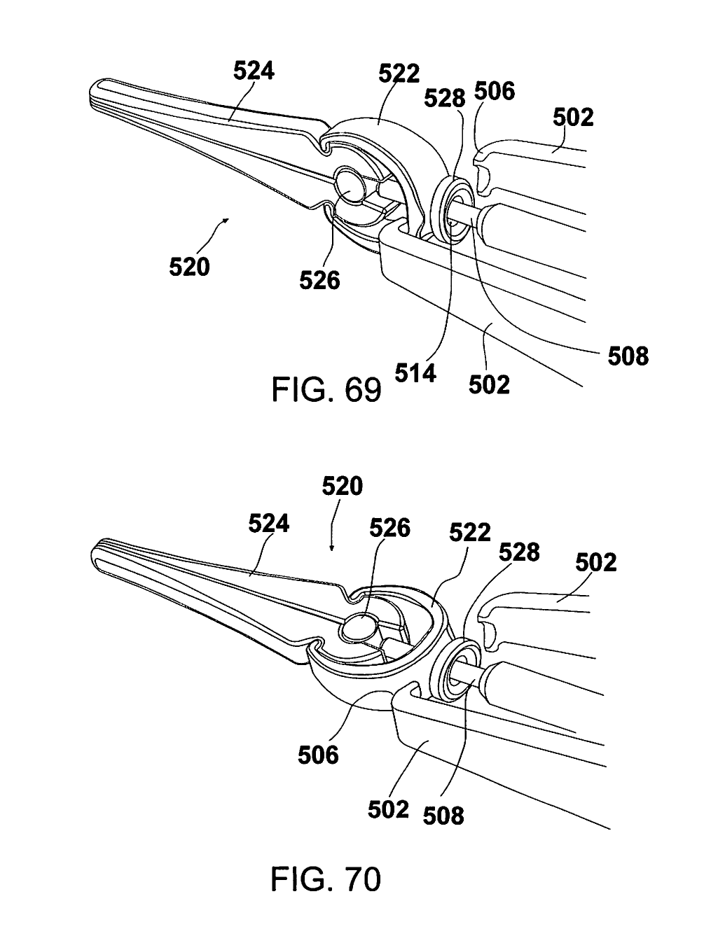

A clip 520 that may be used with the applicator 500 is shown in more detail in FIGS. 66-70, and may be designed according to any of various embodiments of a clip, such as clip 210, with jaws 524, spring 522 and pivot 526. The spring 522 incorporates at least one flange 528 extending outward from the spring 522 between the spring ends 529. In some embodiments, several discrete flanges 528 may be used, or the flange 528 could be circular as shown in FIGS. 69-70. The hooks 506 of the applicator 500 are shaped to engage the flanges 528 for pulling on the flanges 528 when the pin 508 pushes on a portion of the jaws 524 and pivot 526. To operate the applicator 500, with a clip 520, an operator pushes the pin 508 towards the clip 520 through an opening 514 in the spring 522 until the pin 508 engages a portion of the clip 520, preferably the pivot 526. The operator then continues to push the pin 508 forward the hooks 506 swing inward and catch on the flanges 528. The operator may then squeeze the applicator 500, and thereby straighten the leaf springs 510 which push on the push-pin. This opens the jaws by applying a force on the clip pivot toward the jaws and an opposing force on the flanges 528 of the spring 522 to open the jaws 524 of the clip 520. The hooks 506 may be shaped to engage the flange or flanges 528 at discrete positions, or continuously around the clip 520.

An embodiment of an applicator is shown in FIG. 71, with arms 530 connected by pivot 532, and double ended hooks 534 terminating the arms 530. A clip 540 as shown in FIG. 72 with jaws 544 and spring 542 made in accordance with the design of FIG. 210 for example has applicator ends 546 that are shaped to receive the double ended hooks 534 with dual indentations.

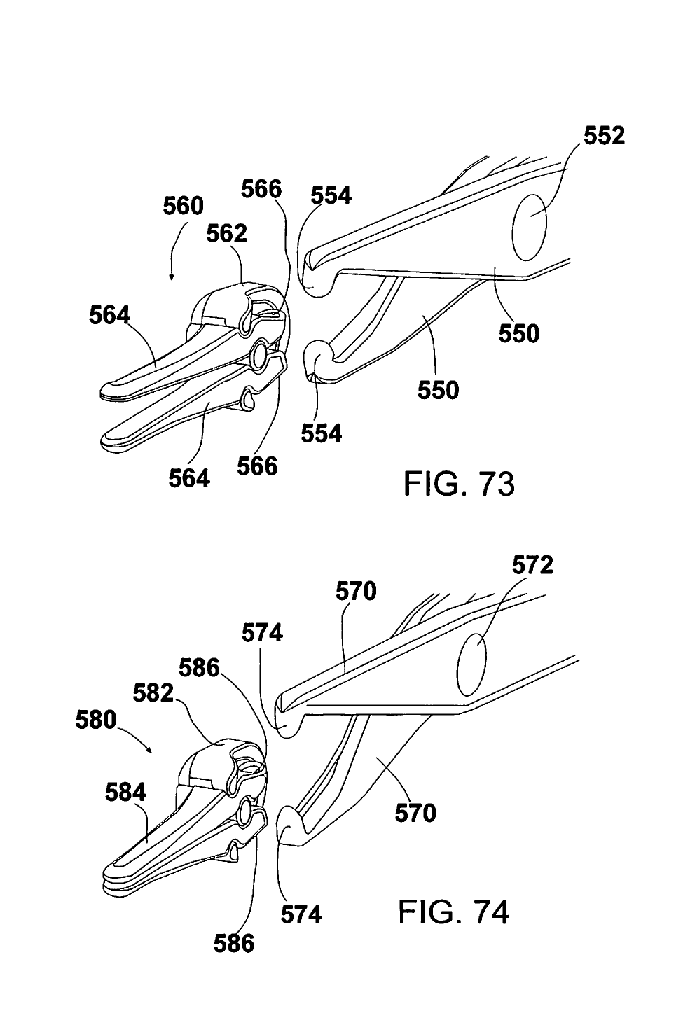

An embodiment of an applicator is shown in FIG. 73, with arms 550 connected by pivot 552, and balls 554 terminating the arms 550. A clip 560 as shown in FIG. 73 with jaws 564 and spring 562 made in accordance with the design of clip 210 for example has applicator ends 566 that are shaped as spherical sockets to receive the balls 554.

An embodiment of an applicator is shown in FIG. 74, with arms 570 connected by pivot 572, and balls 574 terminating the arms 570. A clip 580 as shown in FIG. 74 with jaws 584 and spring 582 made in accordance with the design of clip 210 for example has applicator ends 586 that are shaped as spherical sockets to receive the balls 574.

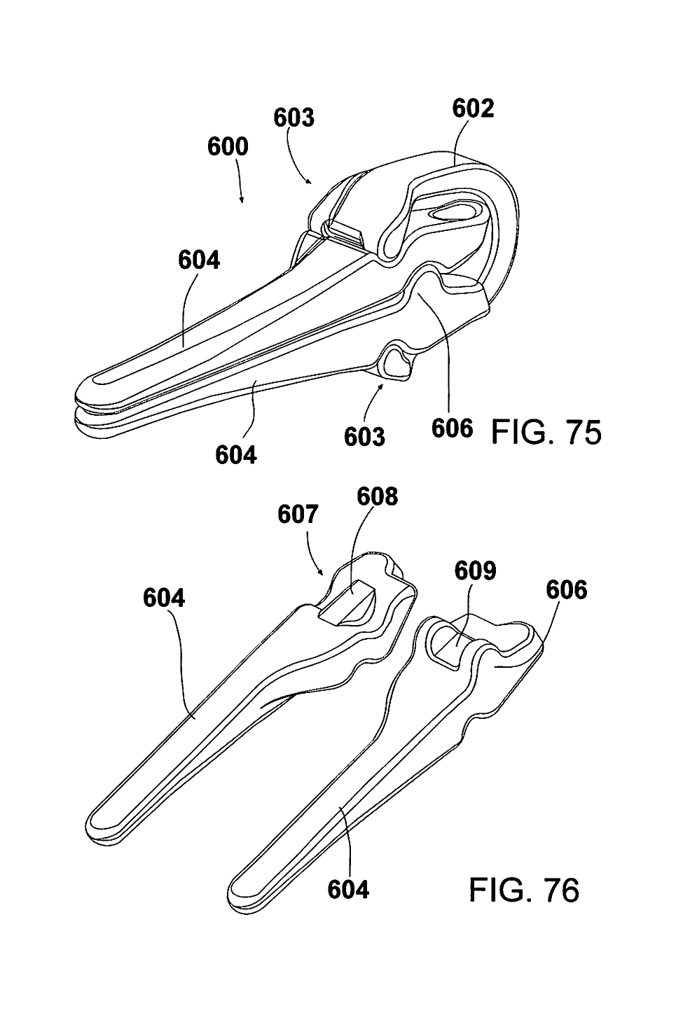

Embodiments of jaws for clips are shown in FIGS. 75-83 that have a pivot integrated into one of the jaws. In FIGS. 75-76, clip 600 has jaws 604 held together by clip 602 that is connected to the jaws 604 by hinge joints 603. The hinge joints 603 function, as with the clip 210, by spring pressure on the hinge joints 603 holding the clip 602 on the jaws 604 and the jaws 604 together. A pivot 606 is formed as part of a jaw 604. The pivot 606 is received by a corresponding socket 607 on the other jaw 604. As with the clip 210, confining walls of socket 607 combined with spring pressure from the spring 602 prevent the clip 602 from becoming detached from the jaws 604. The socket 607 is provided with a tongue or web 608 that cooperates with a groove 609 on the pivot 606 to resist lateral separation of the jaws.

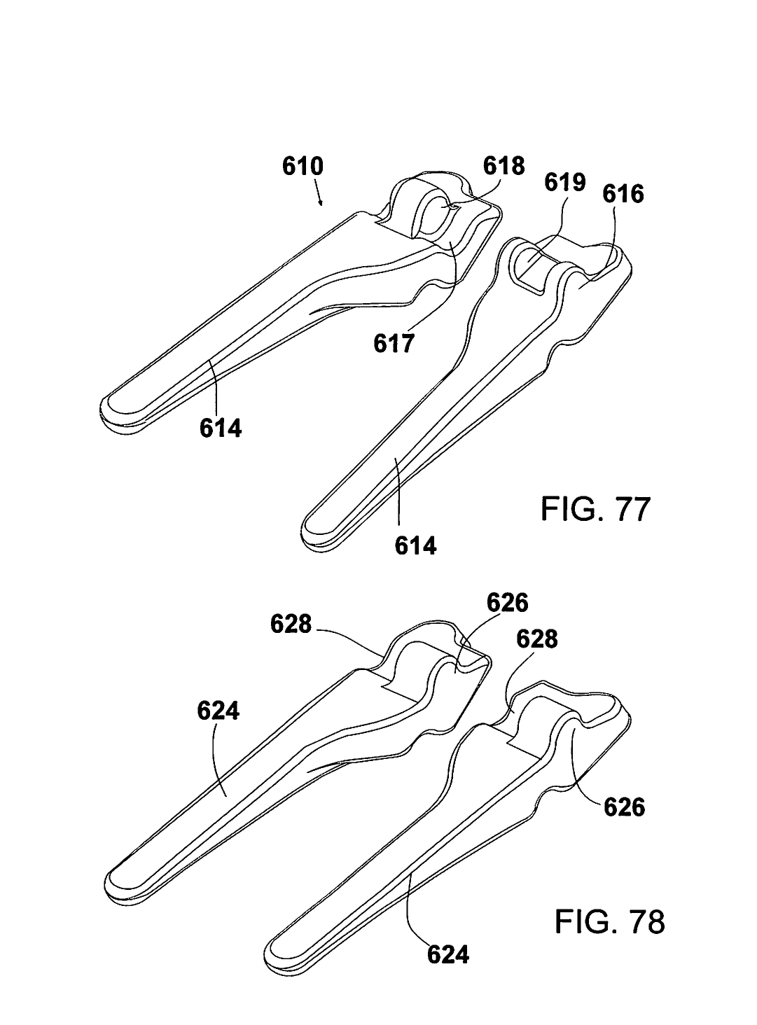

In FIG. 77, clip 610 has jaws 614 held together by spring 612 that is connected to the jaws 614 by hinge joints 613. The hinge joints 613 function, as with the clip 210, by spring pressure on the hinge joints 613 holding the spring 612 on the jaws 614 and the jaws 614 together. A pivot 616 is formed as part of a jaw 614. The pivot 616 is received by a corresponding socket 617 on the other jaw 614. As with the clip 210, confining walls of socket 617 combined with spring pressure from the spring (not shown) prevent the clip 610 from becoming detached from the jaws 614. The socket 617 is provided with a tongue or web 618 that cooperates with a groove 619 on the pivot 616 to resist lateral separation of the jaws. The tongue 618 and groove 619 extend substantially the full depth of the pivot 616.

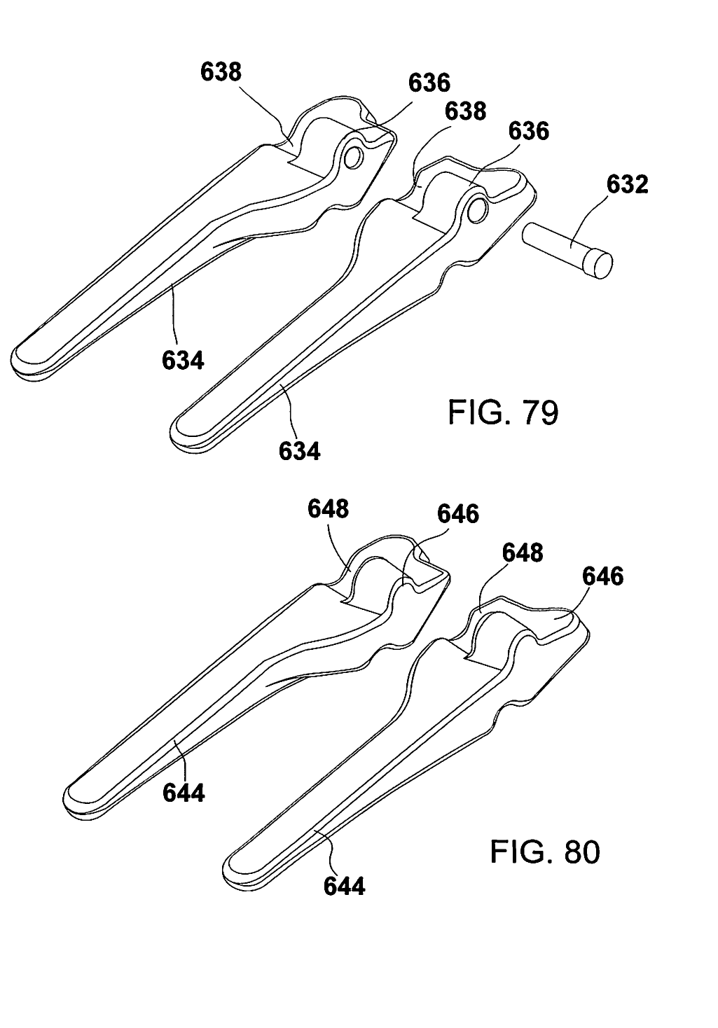

In the design of FIG. 78, jaws 624 are provided each having a portion 626 of a pivot and a portion 628 of a socket integrated with the jaw 624. In this example, half of the pivot and half of the socket is on each jaw. The spring (not shown), but preferably made according to the principles described in relation to clip 210, maintains the lateral stability of the pivot. In FIG. 79, a design similar to the design of FIG. 78, has jaws 634, pivot portions 636, socket portions 638, with the pivot portions 636 connected by a pin 632 that passes through both pivot portions 626. In the design of FIG. 80, jaws 644 are provided each having a conical portion 646 of a pivot and a conical portion 648 of a socket integrated with the jaw 644. In this example, half of the pivot and half of the socket is on each jaw. Each conical portion 646 of the pivot mates with the corresponding conical portion 648 of the socket. The conical portions 646, 648 each widen towards the center of the jaw 644. Pressure from the spring (not shown), but preferably made according to the principles described in relation to clip 210, causes the jaws 644 to be self-centering due to the mating conical pivot and socket combinations. The pivot may also be two points laterally spaced from each other such as two ball protrusions on one jaw and two sockets on the other jaw, or two sockets on each jaw with two separate or connected ball pivots.

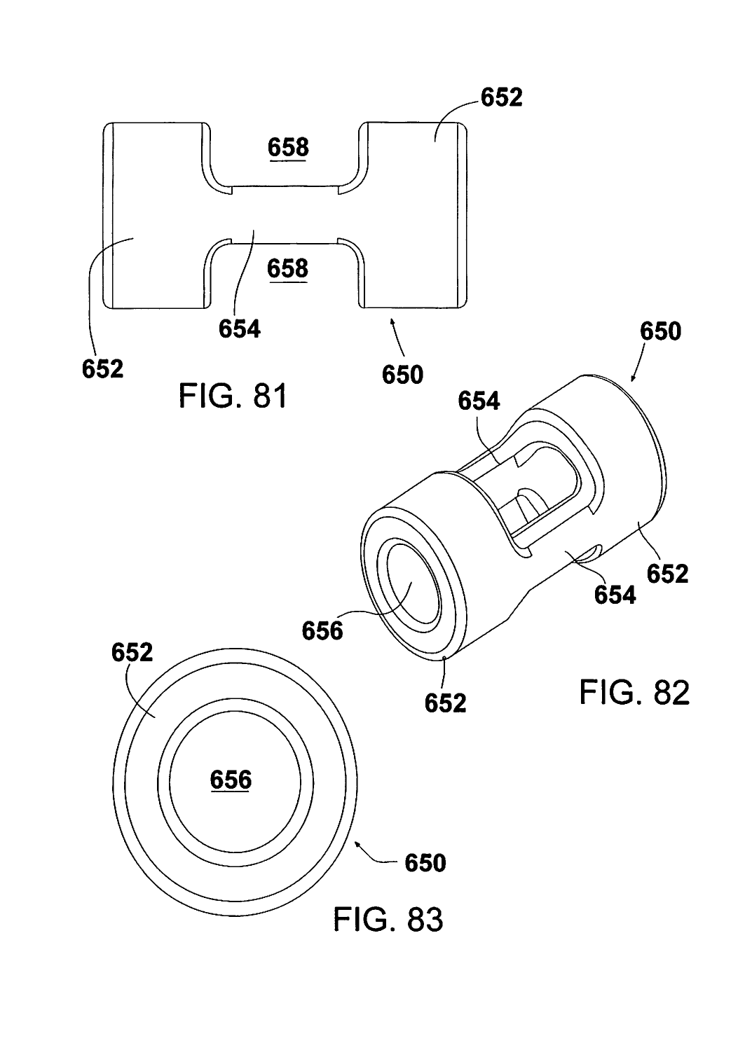

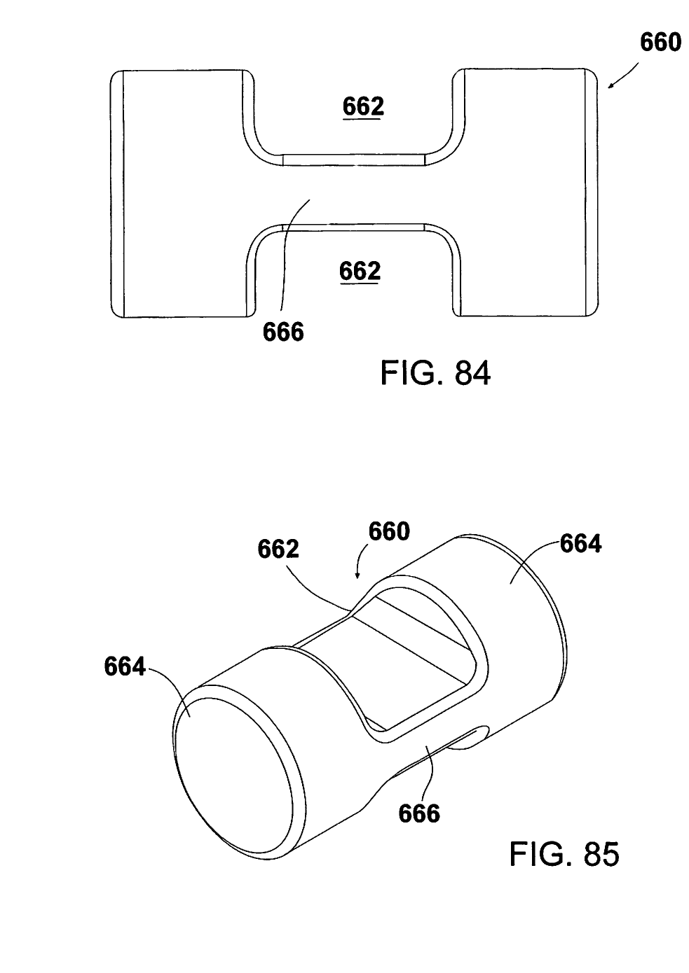

An embodiment of a pivot 650 is shown in FIGS. 81-83 which may be used for example as the pivot 224 in clip 210. The pivot 650 has annular end pieces 652 connected by struts 654. Equivalently, the pivot 650 is formed as a cylinder with a bore 656 and side cut outs 658. With a large enough bore, and if the pivot 650 is made with a ductile metal such as titanium, the pivot can be made to bend under scissoring load and help prevent damage to the jaws. A further example of a pivot 660 is shown in FIGS. 84 and 85 in which a cylindrical piece has cut outs 662, thus providing two cylindrical end pieces 664 connected by a web 666.

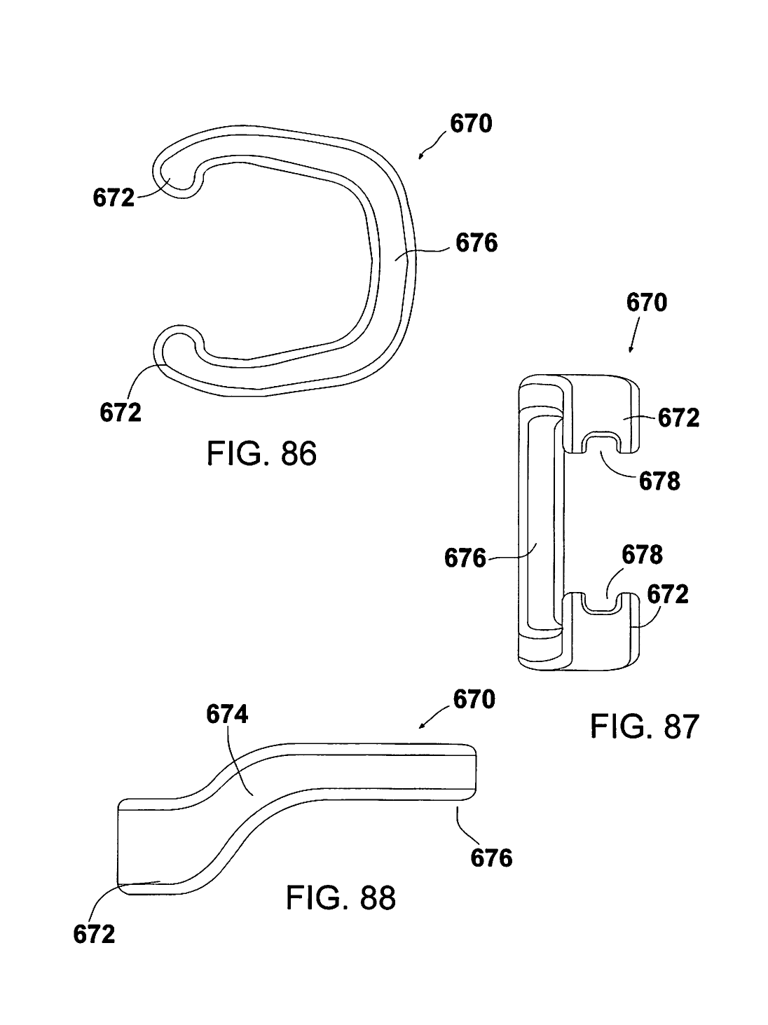

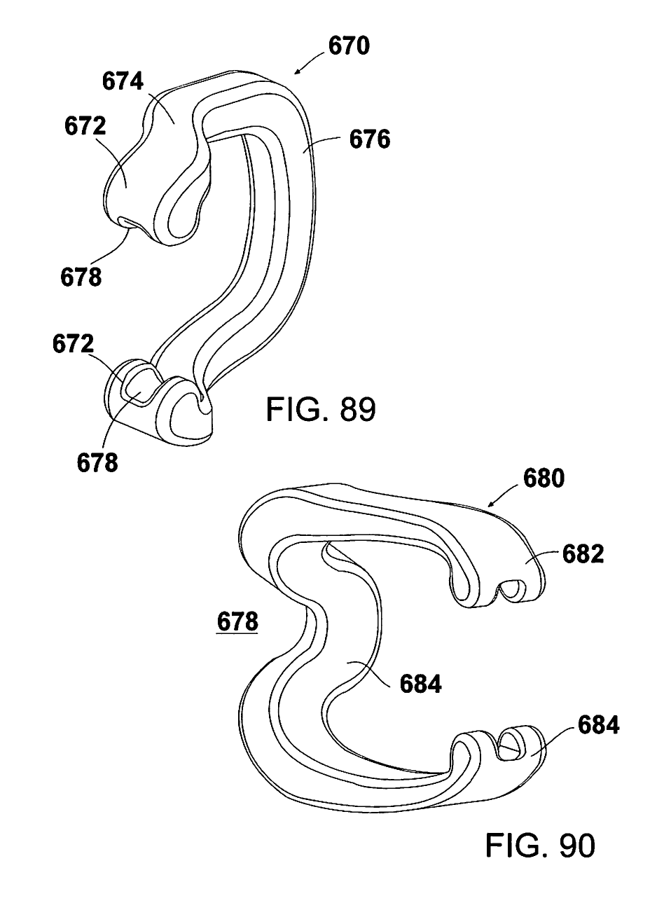

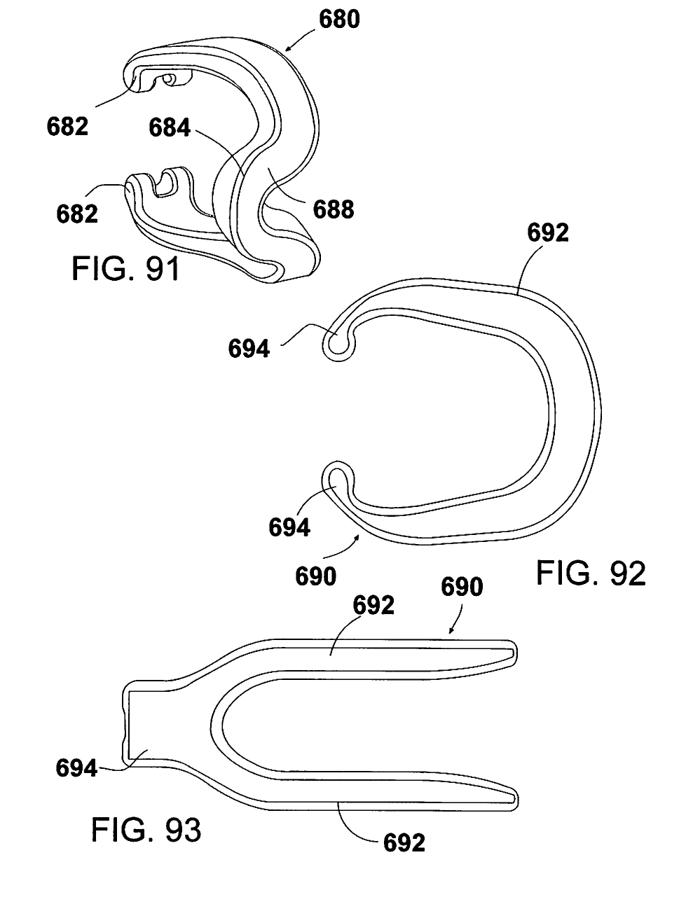

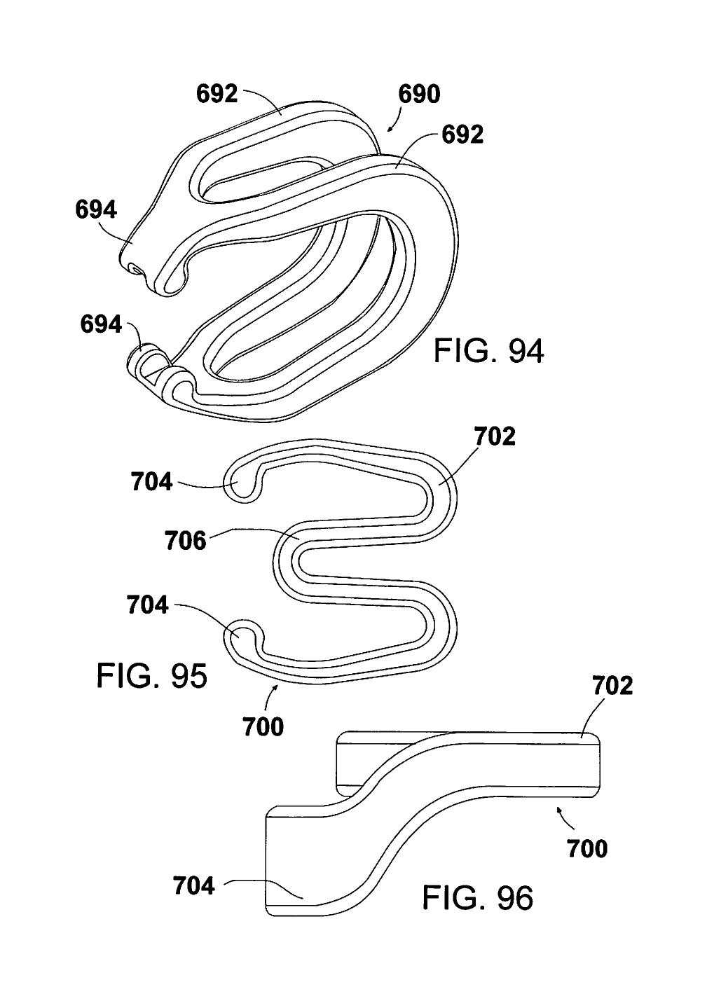

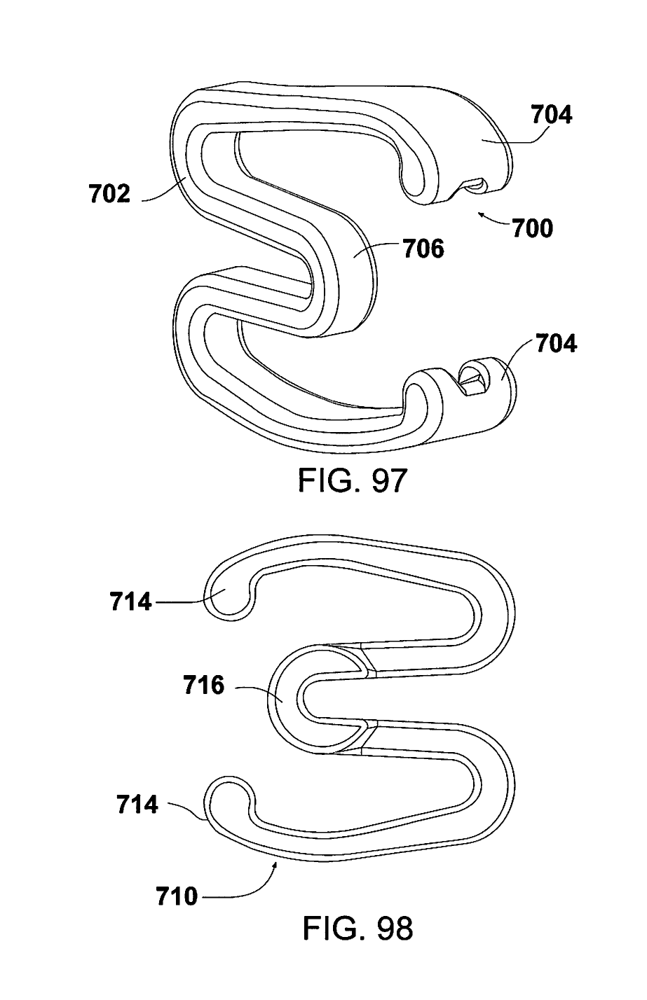

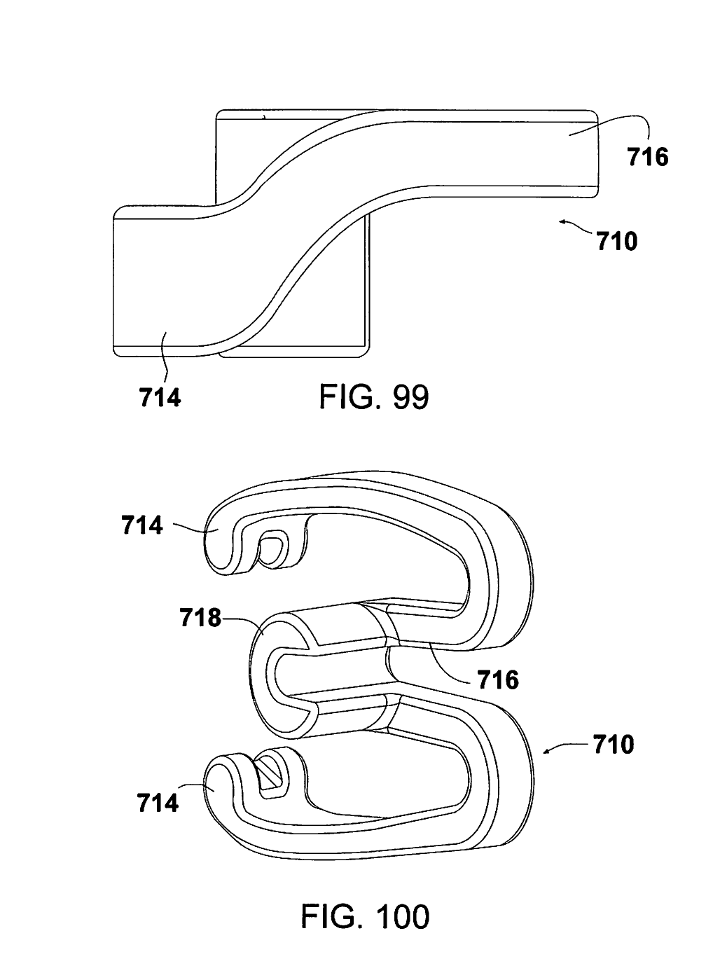

Various embodiments of springs 670, 680, 690, 700, 710 and 720 are shown in FIGS. 86-102. The springs 670-720 may be used in any one of the clips described here. In FIGS. 86-89, spring 670 has spring ends 672 which form part of a hinge joint, an offset portion 674 and rear portion 676. The spring ends 672 have the same width as the jaws to which they are applied, as for example jaws 214 of clip 210. The spring ends 672 are provided with respective grooves 678 to receive for example a tongue or web portion 242 of clip 210. The minimum offset of the spring rear portion 676 in order to clear the jaws (not shown) is therefore equal to the width of the hinge joint. Front end view FIG. 87 shows the offset of the rear portion 676. In the design of FIGS. 90-91, a spring 680 with spring ends 682 has a rear portion 684 that fits into the space behind the jaws (not shown) to which it is applied. The rear portion 684 has a U shaped portion 688 to allow gripping of the applicator ends (not shown) by an applicator. In the embodiment of a spring 690 shown in FIGS. 92-94, the rear of the spring 690 bifurcates rearward of the spring ends 694 to form a pair of spring arms 692. This clip design, while stable, makes it difficult to access the applicator ends of a clip.

A further embodiment of a spring is shown in FIGS. 95-97 in which the spring is designed to fit into the area beside the jaws. Spring 700 has a rear portion 702 between spring ends 704 that has a switched back portion 706. The switched back portion 706 provides additional length of spring without increasing the area over which the spring extends. Spring 710 of FIGS. 98-100 also has a switched back portion 716 between spring ends 714. In spring 710, a pivot 718 is integrated into the switched back portion 716 of the spring 710. The pivot 718 may function for example as the pivot 226 of clip 210. The spring may also be a coiled spring.

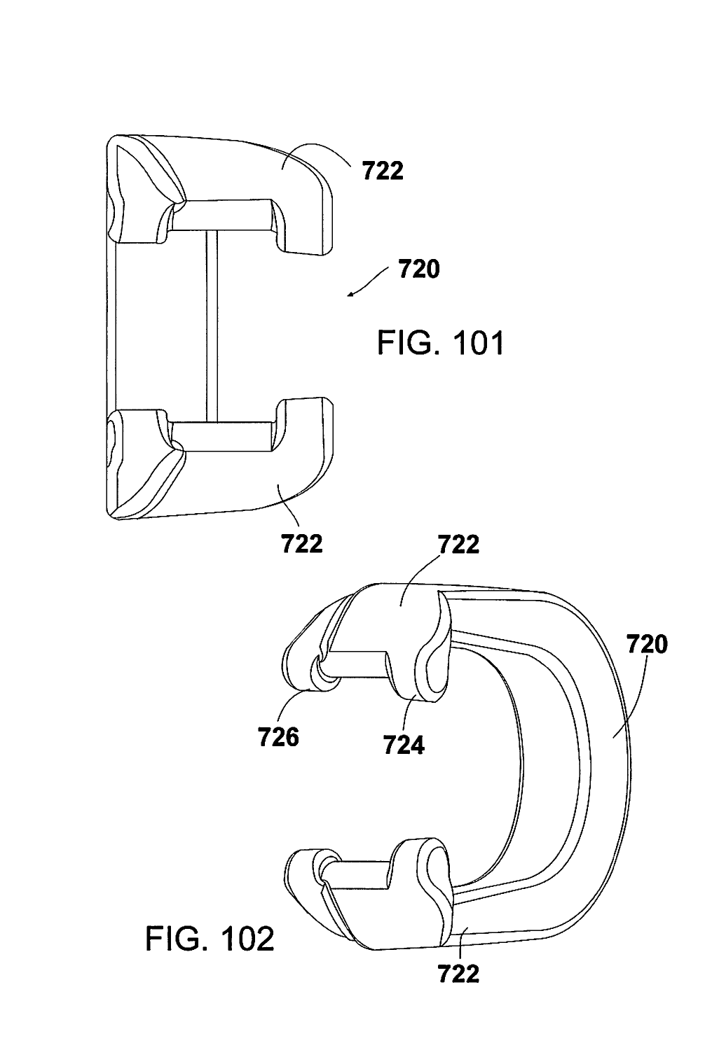

In the embodiment of FIGS. 101-102, spring 720 is pre-twisted or otherwise constructed to provide a uniform spring opening resisting force across the spring ends 722. Due to the offset nature of the spring used for example with a clip 210, the spring may have a tendency to resist opening more readily on the side 726 with the rear portion of the clip. This tendency may be counteracted by various constructions such as providing a stiffer material on the open side 724, or pre-twisting the spring material so that the open side 724 provides a greater spring force than the closed side 726.

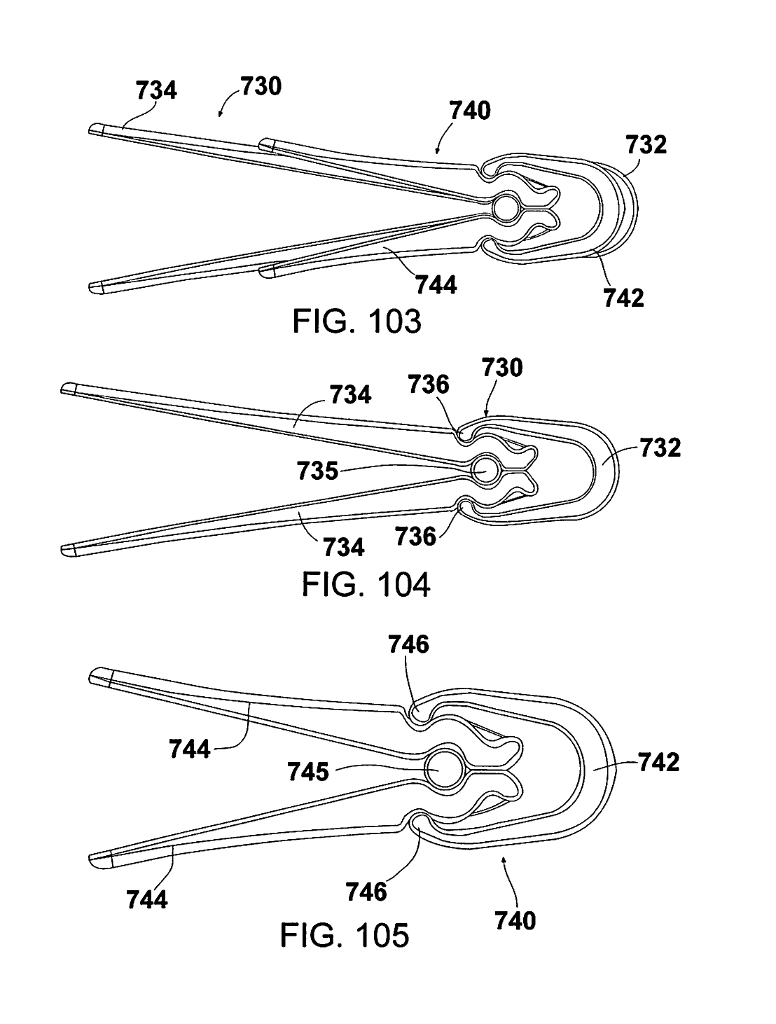

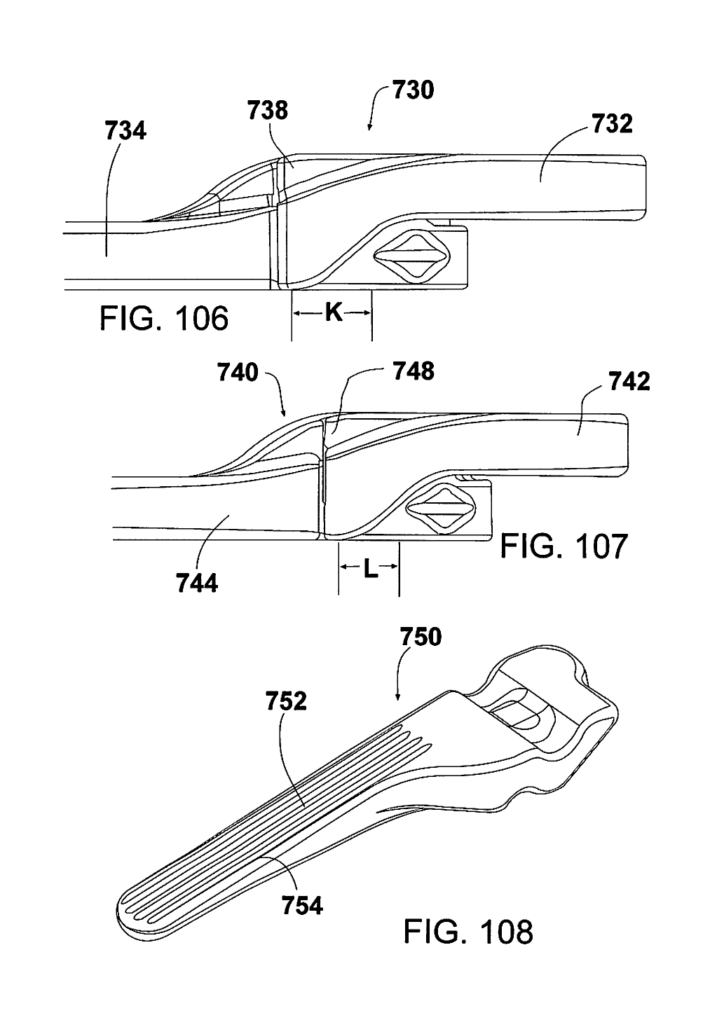

The longer the jaws of a clip, the more force is required from the spring to provide a given surface pressure at the clamping faces, due to increased surface area of the jaws and reduced leverage of the spring. Several methods may be used to avoid enlarging the spring for a clip with short jaws. The spring will always be larger for a longer clip, but with the following considerations, the extra size may be reduced: 1. the jaw faces 223 (see FIG. 1 for example) between the jaw sockets contact at a lower angle on the longer clips, thus requiring less deflection from the spring and allowing a shorter spring than would be necessary for a greater angle. 2, the spring hinge 230 axis is located at a lower angle to the pivot, so it has more leverage on the jaws, which requires less spring force and a “lighter”/thinner cross section spring which is more flexible and can therefore be shorter than would be required for a higher force spring. Thus, for a given spring size, the longer the clip, the less the maximum opening angle, although due to the length of the jaws, the jaws would still open wider than a shorter version of the same clip. Hence, a suitable design of a longer clip in a series of clips would have a maximum opening angle less than the opening angle of the next shorter clip of similar shape, and with a jaw opening size greater than the jaw opening size of the next shorter clip of similar shape. These principles are illustrated in FIGS. 103-105, which show a clip 730 with long jaws 734 in comparison with a clip 740 with short jaws 744 As shown also in FIGS. 106-107, the spring hinge 738 of the clip 730 is further from the pivot 735 (distance K), than the spring hinge 748 of the clip 740 is from the pivot 745 (distance L), that is K>L.

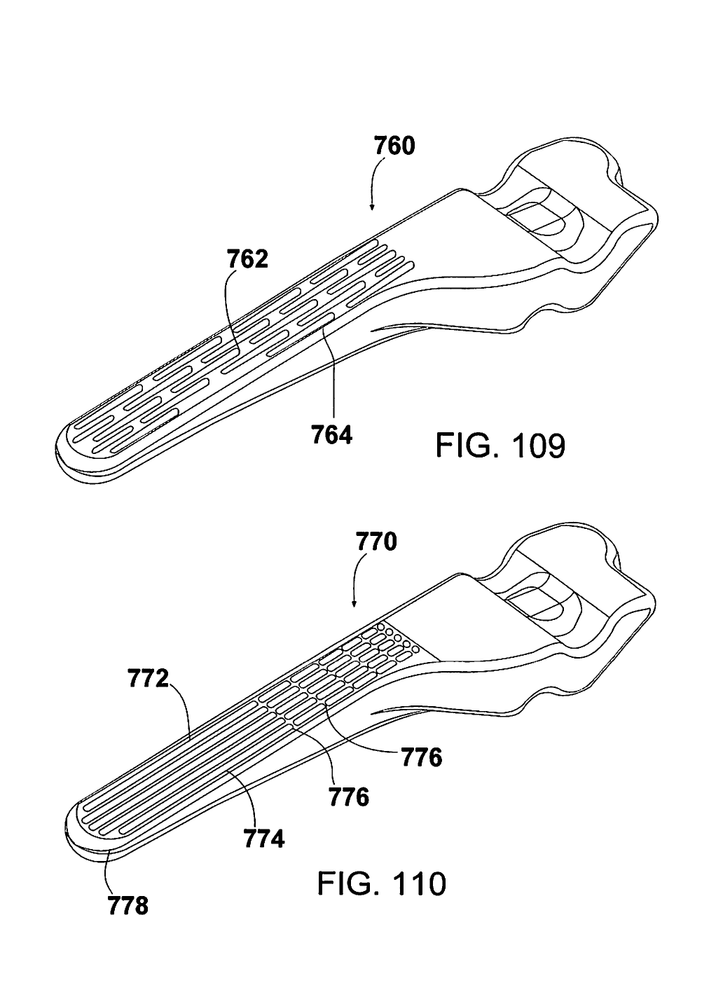

Several clamping face surface structures are shown in FIGS. 108-110. These structures can be used with any of the clips. In FIG. 108, continuous grooves or ridges 752 extend along a major portion of a clamping face 754 of a jaw 750. In FIG. 109, jaw 760 has overlapping grooves or ridges 762 extending along a major portion of a clamping face 764. In FIG. 110, a jaw 770 has overlapping grooves or ridges 772 on a clamping face 774, that are interrupted by one or more areas 776 without structure, or that may comprise a cross groove or ridge. The areas 776 occur where the jaw is thicker, more than half way along the clamping face 774 from the tip 778.

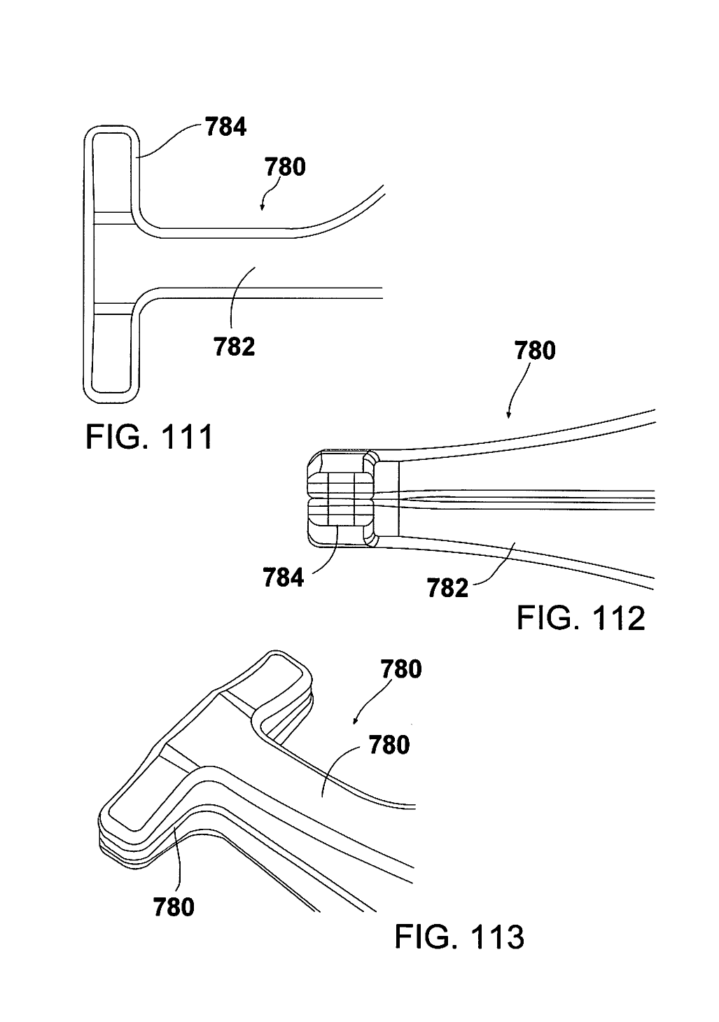

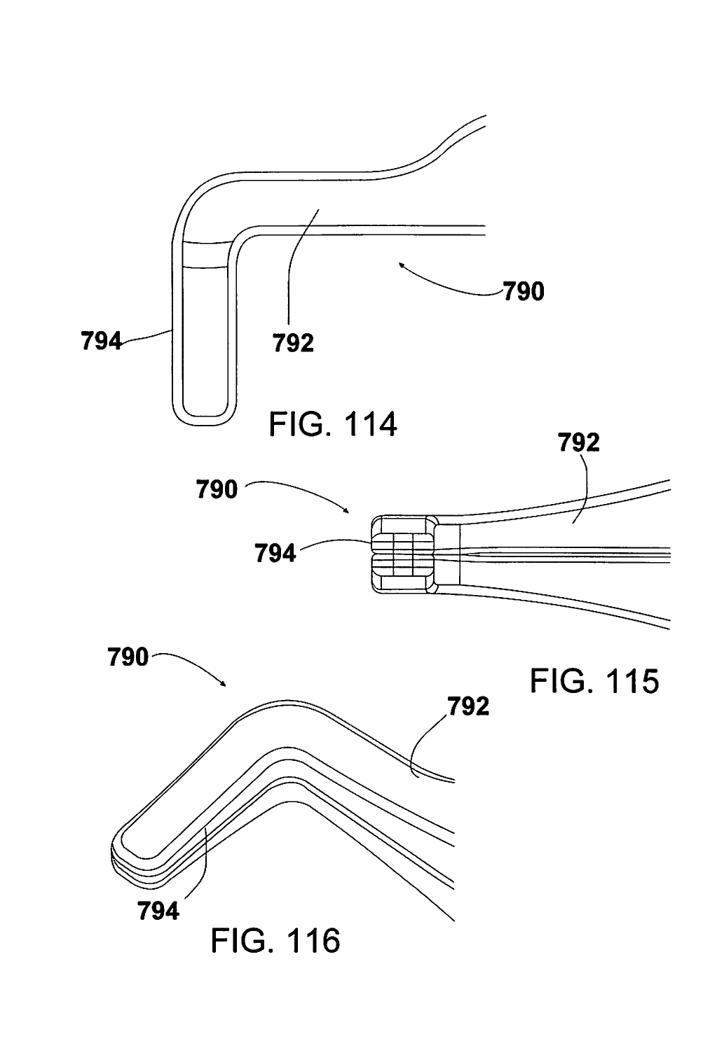

FIGS. 111-113 show an embodiment of a clip 780 with flat jaws 782 that have a T shaped clamping section 784. FIGS. 114-116 show an embodiment of a clip 790 with flat jaws 792 that have an L shaped clamping section 794.

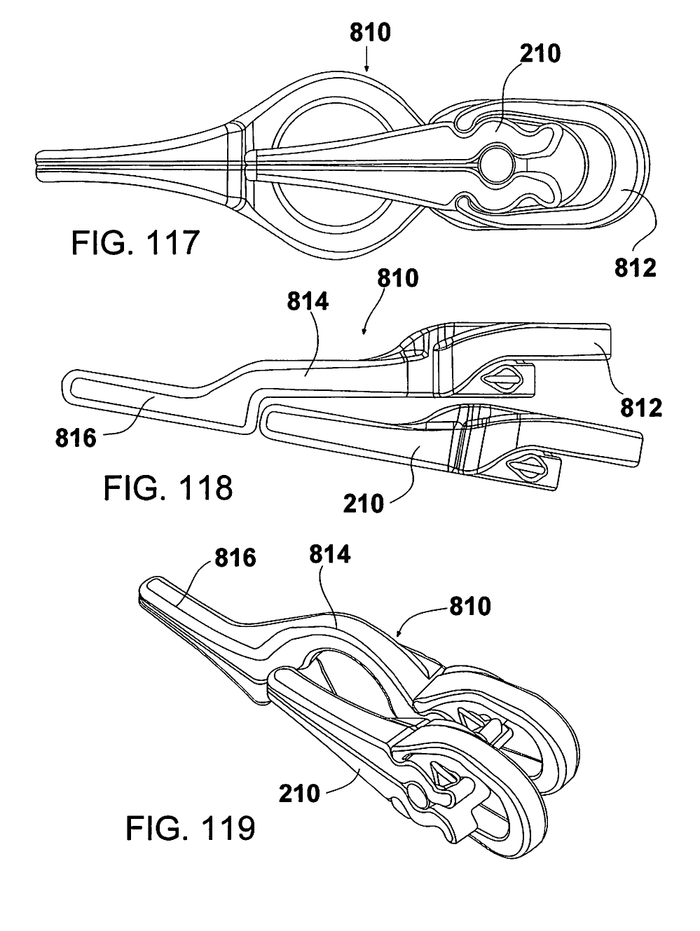

In FIGS. 117-119, a clip 210 is used in combination with a clip 810 with spring 812 and jaws with an initial portion 814, which may be straight, followed by a laterally offset portion 816 that extends forward of the straight portion 814 but off to one side. The clip 810 can be used with various other clips beside the clip 210 to provide for a longer and more complete occlusion of an aneurysm. The straight portion 814 is shown as circular in a plane perpendicular to the pivot axis, but could be flat. The degree of offset is sufficient to accommodate a cooperating clip. The offset portion may or may not be parallel to the initial straight portion (and may or may not be straight).

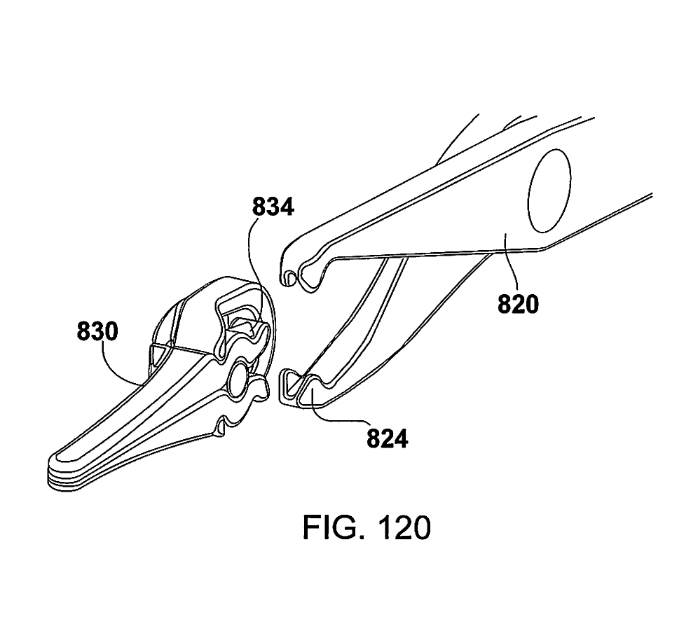

In FIG. 120, an applicator 820 has pyramidal applicator tip ends 824 that engage with correspondingly shaped applicator contact areas 834 of clip 830. The clip 830 is otherwise designed according to the clip 210.

The jaws may be a low reflectivity gray in color. Low reflectivity is desirable for the clip because it will be used under the intense illumination of surgical lighting. The titanium springs may be color coded to differentiate between permanent and temporary (preferably purple for permanent and gold for temporary). The titanium springs are therefore preferably a non-smooth finish such as a glass bead-blasted finish. The ceramic jaws have a preferably smooth finish for strength and reduced wear against the titanium but are preferably gray in color to reduce reflectivity.

Immaterial modifications may be made to the embodiments described here without departing from what is covered by the claims.

1. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
respective joints formed between each spring end and the corresponding jaw, each joint comprising a hinge joint having a hinge axis.

2. The surgical clip of claim 1 in which each hinge joint comprises confining walls, the confining walls having sloped contact faces, the sloped contact faces being angled at between 140 degrees and 40 degrees to each other.

3. The surgical clip of claim 2 in which the sloped contact faces of each hinge joint are angled at between 80 degrees and 100 degrees to each other.

4. The surgical clip of claim 1 in which the confining walls of each hinge joint are formed as part of a respective one of the jaws.

5. The surgical clip of claim 1 in which contact points between the spring and the jaws at each hinge joint are distributed spatially in at least two dimensions, one dimension being parallel to the hinge axis and another dimension being perpendicular to the hinge axis.

6. The surgical clip of claim 5 in which the contact points at each hinge joint are spread parallel to the hinge axis by a greater amount than the contact points are spread perpendicular to the hinge axis.

7. The surgical clip of claim 1 in which each hinge joint comprises a tongue and groove structure oriented to oppose relative motion of the spring and jaws parallel to the hinge axis.

8. The surgical clip of claim 1 in which the hinge joints are located with respect to the pivot so that lines joining centers of each hinge joint to the pivot axis form an angle of greater than 90 degrees.

9. The surgical clip of claim 5 in which:
the spring ends having an initial pre-load, where the initial pre-load is the difference between a resting separation where the spring ends are not loaded and an initial loaded separation when the spring is engaged with the jaws and the jaws are in contact;
at least one of A or B is present where A is that each hinge joint has a hinge axis, and the contact points at each hinge joint are distributed about a hinge gap between the respective spring end and respective jaw, the hinge gap extending parallel to the hinge axis, and B is that the pivot contacts the jaws at one or more sets of pivot contact points, and the pivot contact points of each set of pivot contact points are distributed about at least one pivot gap between the pivot and the jaws, the pivot gap extending parallel to the pivot axis; and
each of the hinge gaps and pivot gap or pivot gaps have a thickness, and the sum of the thicknesses of the hinge gap and the pivot gap or pivot gaps is less than the initial pre-load.

10. The surgical clip of claim 1 in which each applicator end has an applicator contact area, and a portion of the spring between the hinge joints is offset from planes perpendicular to the pivot axis that pass through the applicator contact areas to allow visibility and accessibility of the applicator contact areas from directly behind the surgical clip.

11. The surgical clip of claim 1 in which each hinge axis is parallel to the pivot axis.

12. The surgical clip of claim 1 in which each applicator end comprises a ball or socket.

13. The surgical clip of claim 1 in which each applicator end comprises a socket, the socket has a side opposed to the pivot and the socket has a raised lip at the side opposed to the pivot.

14. The surgical clip of claim 1 in which the jaws are formed at least in part of one or more MRI invisible materials and the spring is formed at least in part of one or more MRI compatible metals.

15. The surgical clip of claim 14 in which the jaws are made of ceramic.

16. The surgical clip of claim 1 in which the applicator ends comprise matching applicator end to applicator end contact surfaces.

17. The surgical clip of claim 1 in which the hinge joints are wider in a direction parallel to the pivot axis than the jaws are wide at the clamping faces of the jaws.

18. The surgical clip of claim 1 in which the pivot is wider in a direction parallel to the pivot axis than the jaws are wide at the clamping faces of the jaws.

19. The surgical clip of claim 1 in which the pivot separates the jaws so that, when at least a portion of the clamping faces of the jaws are parallel, the clamping faces are spaced from each other.

20. The surgical clip of claim 1 in which the clamping faces each have a divergent portion, so that, when at least a portion of the clamping faces of the jaws are parallel, the divergent portions diverge away from each other, the divergence being the greater closer to the pivot.

21. The surgical clip of claim 1 in which the jaws taper towards the clamping ends when measured in a direction perpendicular to the pivot axis and perpendicular to a plane bisecting the jaws.

22. The surgical clip of claim 1 in which the clamping faces each have a long dimension and a short dimension, and the long dimension is parallel to the pivot axis.

23. The surgical clip of claim 22 in which the jaws form an L or T shape.

24. The surgical clip of claim 22 in which the jaws form a T shape.

25. The surgical clip of claim 24 in which the jaws have portions forming arms of the T shape, and the arms are offset from each other in a direction perpendicular to the pivot axis to form a stitch clip.

26. The surgical clip of claim 1 in which the jaws each separate from each other to form respective arches in a portion between the clamping faces and the pivot.

27. The surgical clip of claim 26 in which the respective arches combine to form a circular jaw portion.

28. The surgical clip of claim 27 in which the jaws are L or T shaped, and the clamping faces are formed as part of an arm or arms of the L or T shape.

29. The surgical clip of claim 27 in which the clamping faces have a long dimension and a short dimension and the long dimension extends radially in relation to the circular jaw portion.

30. The surgical clip of claim 1 in which one of the jaws is shorter than the other jaw, and the clamping faces form a non-zero angle to a plane passing through the pivot axis and bisecting the spring.

31. The surgical clip of claim 1 in which the spring incorporates at least one flange extending outward from the spring between the spring ends.

32. The surgical clip of claim 1 in which the pivot is seated between confining walls of at least one of the jaws, and the confining walls are angled at between 40 and 140 degrees to each other.

33. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the jaws being formed at least in part of one or more MRI invisible materials and the spring being formed at least in part of one or more MRI compatible metals.

34. The surgical clip of claim 33 in which the jaws are made of ceramic.

35. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the pivot being received by confining walls in at least one of the jaws, the confining walls having sloped contact faces, the sloped contact faces being angled at between 140 degrees and 40 degrees to each other.

36. The surgical clip of claim 35 in which the sloped contact faces of the confining walls that receive the pivot are angled at between 80 degrees and 100 degrees to each other.

37. The surgical clip of claim 35 in which a tongue and groove structure oriented to oppose relative motion of the spring and jaws parallel to the pivot axis is formed between the spring and at least one jaw.

38. The surgical clip of claim 35 in which the pivot contacts the at least one jaw at contact points, and the contact points are distributed about a gap between the pivot and the at least one jaw, the gap extending parallel to the pivot axis.

39. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
each applicator end comprises a ball or socket.

40. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the pivot being restrained from twisting motion in relation to the jaws by confining walls formed in one of the jaws and the pivot.

41. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the pivot being wider in a direction parallel to the pivot axis than the jaws are wide at the clamping faces of the jaws.

42. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the pivot separating the jaws so that, when at least a portion of the clamping faces of the jaws are parallel, the clamping faces are spaced from each other.

43. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the clamping faces each having a divergent portion, so that, when at least a portion of the clamping faces of the jaws are parallel, the divergent portions diverge away from each other, the divergence being greater the closer to the pivot.

44. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the jaws tapering towards the clamping ends when measured in a direction perpendicular to the pivot axis.

45. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the clamping faces each having a long dimension and a short dimension, and the long dimension being parallel to the pivot axis.

46. The surgical clip of claim 45 in which the jaws form an L or T shape.

47. The surgical clip of claim 46 in which the jaws form a T shape.

48. The surgical clip of claim 47 in which the jaws have portions forming arms of the T shape, and the arms are offset from each other in a direction perpendicular to the pivot axis.

49. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the jaws each separating from each other to form respective arches in a portion between the clamping faces and the pivot.

50. The surgical clip of claim 49 in which the respective arches combine to form a circular jaw portion.

51. The surgical clip of claim 50 in which the jaws are L or T shaped, and the clamping faces are formed as part of an arm or arms of the L or T shape.

52. The surgical clip of claim 50 in which the clamping faces have a long dimension and a short dimension and the long dimension extends radially in relation to the circular jaw portion.

53. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
one of the jaws being shorter than the other jaw, the clamping faces forming a non-zero angle to a plane passing through the pivot axis and bisecting the spring.

54. A surgical clip, comprising:
a pair of jaws, each jaw having a clamping end, a clamping face and an applicator end;
a clip pivot between the jaws, the clip pivot having a pivot axis about which the jaws pivot;
a spring having spring ends, each spring end bearing against a corresponding one of the jaws between the clamping end of the jaw and the pivot; and
the spring incorporating at least one flange extending outward from the spring between the spring ends.

55. A medical applicator and clip combination, comprising:
a clip having pivoting jaws held together by a clip spring, each jaw having an applicator end;
the applicator having pivoting arms held apart by an applicator spring, each arm having a tip end; and
at least a portion of a side of the jaws adjacent the applicator being coplanar with at least a portion of a side of the applicator adjacent the clip when the applicator tip ends are engaged with the clip applicator ends.

56. A medical applicator, comprising:
a pair of arms connected by a pivot having a pivot axis, each arm of the pair of arms having a tip end on one side of the pivot and a handle on the other side of the pivot;
a spring between the arms, the spring being attached to at least one of the arms and, in operation, urging the arms apart; and
each handle having an inner portion facing the other handle, the inner portions incorporating a grip surface.

57. A medical applicator, comprising:
a pair of arms connected by a pivot having a pivot axis, each arm of the pair of arms having a tip end on one side of the pivot and a handle on the other side of the pivot;
a spring between the arms, the spring being attached to at least one of the arms and, in operation, urging the arms apart; and
a latch secured to one handle by a latch pivot, a catch on the other handle, the latch pivot extending fully across the one handle to resist bending of the latch.

58. A medical applicator, comprising:
a pair of arms connected by a pivot having a pivot axis, each arm of the pair of arms having a tip end on one side of the pivot and a handle on the other side of the pivot;
an applicator spring between the arms, the applicator spring being attached to at least one of the arms and, in operation, urging the arms apart; and
a latch secured to a first one of the handles by a latch pivot, a catch on the second one of the handles, the latch being biased by a latch spring towards the first handle.

59. The medical applicator of claim 58 in which the latch has a latching end with a hook, the hook having a slanted inwardly widening exterior face.

60. The medical applicator of claim 58 further comprising a stop structure on the first handle, the stop structure preventing contact of the latching end of the latch with the first handle.

61. The medical applicator of claim 60 in which the stop structure comprises a pin secured to the first handle adjacent the latch pivot.

62. The medical applicator of claim 58 further comprising a stop structure preventing rotation of the latch by more than a pre-defined distance beyond the catch.

63. The medical applicator of claim 60 in which the stop structure comprises a pin and one or more shoulders on the latch.

64. The medical applicator of claim 55 in which each tip end is formed as either a ball or socket.

65. A medical applicator, comprising:
a pair of arms connected by a pivot having a pivot axis, each arm of the pair of arms having a tip end on one side of the pivot and a handle on the other side of the pivot;
a spring between the arms, the spring being attached to at least one of the arms and, in operation, urging the arms apart; and
each tip end being formed as either a ball or socket.

66. A medical applicator, comprising:
at least a first handle and a second handle connected to each other at an applicator pivot, each of the at least a first handle and second handle having respective hooks at portions spaced from the applicator pivot;
a pin extending away from the applicator pivot, and attached to each of the at least a first handle and second handle by respective lever arms, the lever arms being pivotally attached to each of the pin and the at least a first handle and the second handle between the respective hooks and the applicator pivot;
the pin extending further away from the applicator pivot than the at least a first handle and the second handle extend away from the applicator pivot; and
the hooks each having inwardly extending hook arms.

67. A surgical clip and applicator combination, comprising:
a surgical clip, said surgical clip comprising:
a pair of jaws, each jaw having a clamping end, a clamping face and an applicator end;
a clip pivot between the jaws, the clip pivot having a pivot axis about which the jaws pivot;
a spring having spring ends, each spring end bearing against a corresponding one of the jaws between the clamping end of the jaw and the pivot;
the spring incorporating at least one flange extending outward from the spring between the spring ends; and
a second flange extending outward from the spring between the spring ends; and
a medical applicator, said medical applicator comprising:
at least a first handle and a second handle connected to each other at an applicator pivot, each of the at least a first handle and second handle having respective hooks at portions spaced from the applicator pivot;
a pin extending away from the applicator pivot, and attached to each of the at least a first handle and second handle by respective lever arms, the lever arms being pivotally attached to each of the pin and the at least a first handle and the second handle between the respective hooks and the applicator pivot;
the pin extending further away from the applicator pivot than the at least a first handle and the second handle extend away from the applicator pivot; and
the hooks each having inwardly extending hook arms, the hooks being shaped to engage the flanges for pulling on the flanges when the pin pushes on a portion of the jaws or pivot.

68. A method of latching a medical applicator onto a clip having a clip spring, where the medical applicator is formed according to claim 58, the method comprising the steps of:
closing the first handle and the second handle against force provided by at least one of the applicator spring and the clip spring;
moving the latch against force provided by at least one of the latch spring and the clip spring until the latch abuts against a stop structure;
continuing to close the first handle and the second handle together while pressing the latch against the stop structure or catch as the hook engages the catch until the hook latches onto the catch.

69. The surgical clip of claim 1 in which the spring is constructed to provide a uniform spring opening resisting force across the spring ends in at least one closing position of the spring.

70. The surgical clip of claim 1 in which at least a portion of at least one of the clamping faces of the jaws is provided with a structured surface.

71. The surgical clip of claim 70 in which the structured surface comprises grooves or ridges.

72. The surgical clip of claim 71 in which the grooves or ridges are continuous along the at least one of the clamping faces.

73. The surgical clip of claim 71 in which the grooves or ridges are overlapping.

74. The surgical clip of claim 70 in which the structured surface is interrupted at a location at least further than half-way along the at least one of the clamping faces from the clamping end of the jaws.

75. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
a portion of at least one of the clamping faces of the jaws is provided with a structured surface.

76. The surgical clip of claim 75 in which the structured surface comprises grooves or ridges.

77. The surgical clip of claim 76 in which the grooves or ridges are continuous along the at least one of the clamping faces.

78. The surgical clip of claim 76 in which the grooves or ridges are overlapping.

79. The surgical clip of claim 75 in which the structured surface is interrupted at a location at least further than half-way along the at least one of the clamping faces from the clamping end of the jaws.

80. A surgical clip, comprising:
a pair of jaws, each jaw having a clamping end, a clamping face and an applicator end;
a pivot between the jaws, the pivot having a pivot axis about which the jaws pivot;
a spring having spring ends, each spring end bearing against a corresponding one of the jaws between the clamping end of the jaw and the pivot; and
the jaws having an initial straight portion and a laterally offset clamping portion, the laterally offset clamping portion extending forwardly of the initial straight portion.

81. The surgical clip of claim 1 in which the jaws have an initial portion and a laterally offset clamping portion, the laterally offset clamping portion extending forwardly of the initial portion.

82. A surgical clip having a pair of jaws, pivoted together, with a spring holding the jaws together, the surgical clip comprising one or more of the following features:
the spring contacting the jaws at hinge joints;
the spring contacting the jaws at hinge joints having confining walls with sloped contact faces at various angles;
contact points of the spring with the jaws being spatially distributed;
the spring contacting the jaws at hinge joints with a tongue and groove structure in the hinge joints for centering of the spring;
spring ends to pivot angle being larger than 90 degrees;
the spring contacting the jaws at hinge joints with a center gap in the hinge joint between spring ends and the jaws;
an offset rear portion (applicator end) of the jaws for receiving an applicator;
an offset rear portion (applicator end) of the jaws for receiving an applicator, the jaws having a ball or socket at an applicator end for connection to an applicator;
an offset rear portion (applicator end) of the jaws for receiving an applicator, a flange at the back of the applicator end;
the jaws being formed of an MRI invisible material and the spring being made of an MRI compatible material; and
an offset rear portion (applicator end) of the jaws for receiving an applicator, the applicator ends having matching surfaces that contact each other when the jaws are opened.

83. The surgical clip of claim 82 in which the polyetheretherketone is fiber reinforced.

84. The surgical clip of claim 1 in which the jaws have a divergent portion forming at least one polygonal arch.

85. The surgical clip of claim 1, in which the hinge axis of at least one hinge joint is not parallel to the pivot axis.

86. The surgical clip of claim 85 in which the hinge axis at least one hinge joint is at an angle of less than 45 degrees to the pivot axis.

87. The surgical clip of claim 85 in which the hinge axis at least one hinge joint is at an angle of less than 30 degrees to the pivot axis.

88. The surgical clip of claim 87 in which the hinge axis of each hinge joints is at an angle of less than 30 degrees to the pivot axis.

89. The surgical clip of claim 1 in which the jaws comprise polyetheretherketone.
